(19) United States
(12) Patent Application Publication
(10) Pub. No.: US 2002/0023277 A1
(43) Pub. Date: Feb. 21, 2002

(54) Vascular endothelial zinc finger 1 gene and protein and uses thereof

(76) Inventors: HEIDI STUHLMANN, NEW YORK, NY (US); JING-WEI XIONG, NEW YORK, NY (US); MARK B. TAUBMAN, LARCHMONT, NY (US)

Correspondence Address: BAKER & BOTTS, 30 ROCKEFELLER PLAZA, NEW YORK, NY, 10112

(21) Appl. No.: 09/083,290

(22) Filed: May 22, 1998

(51) Int. Cl. A01K 67/00; C07H 21/04; C12P 21/02

(52) U.S. Cl. 800/8; 800/9; 435/69.1; 435/320.1; 536/23.1; 536/23.5; 536/24.1

(57) ABSTRACT

The present invention relates to the diagnosis and treatment of vascular disorders, and to assays for the identification of agents which act upon the circulatory system. It is based, at least in part, on the identification of a novel mouse gene, termed Vezf1 (for “Vascular endothelial zinc finger 1”), which is expressed at higher levels during embryonic development of the circulatory system, in damaged blood vessels, and in newly formed blood vessels associated with tumor growth.

### 2. BACKGROUND OF THE INVENTION

### 2.1. Foramtion of Blood Vessels During Embryonic Development

The embryonic vascular system develops by two distinct processes, termed vasculogenesis and angiogenesis (Breier and Risau, 1996, Trends Cell Biol.6:454-456; Folkman and D'Amore, 1996, Cell 87:1153-1155; Risau, 1997, Nature 386:671-674). Both are complex multistep processes involving remodeling of extracellular matrix and regulating the proliferation, migration, differentiation, and assembly of endothelial cells to produce, modify, and maintain the complex networks of arteries and veins which constitute the circulatory system. “asculogenesis” is the term applied to the process whereby endothelial cells differentiate de novo from mesodermally derived precursors in the embryo and assemble into primitive blood vessels. These precursor cells, called “angioblasts”, are vascular endothelial cells which have not yet organized to form a lumen. Genetic and developmental studies suggest the existence of a bipotential precursor cell, the “hemangioblast”, that gives rise to both angioblasts and hematopoietic cells (Kennedy et al., 1997, Nature 386:488-492; Shalaby et al., 1995, Nature 376: 62-66). In vertebrates, angioblasts first arise at the early headfold stage in the extraembryonic yolk sac. These angioblasts form by aggregation of splanchnic mesodermal cells into angiogenic clusters, or “blood islands”. They, in turn, differentiate into double-walled tubes having an inner wall formed by flat endothelial cells, which create a cellular lining for the resulting blood vessel, and an outer wall formed by smooth muscle cells. The central cells of the blood islands differentiate into embryonic hematopoietic cells. Differentiation and growth of the blood islands eventually give rise to a capillary network that drains into the two vitelline veins of the embryo. In addition, vasculogenesis also occurs in the embryo itself (as opposed to the yolk sac). Angioblasts arise in the paraxial and lateral mesoderm of the embryo and give rise to primordia of the heart, the dorsal aorta, and large vessels of the head, lung, and gastrointestinal system.

“Angiogenesis” is a process involving the maturation and remodelling of the primitive vascular plexus into a complex network of large and small vessels. In addition, angiogenesis leads to sprouting of pre-existing vessels into intersomitic spaces and initially avascular organs, such as kidney, brain, and limb buds (Coffin and Poole, 1991, Anat. Rec. 231:383-395).

### 2.2. Adult Angiogenesis

After the circulatory system has been fully formed, angiogenesis is required for the normal growth of postnatal tissues, and continues throughout adult life, accounting for transient phases of neo-vascularization during the normal female menstrual cycle, during pregnancy in the placenta, and during wound healing (Folkman, 1995, Nature Med. 1: 27-31; Folkman and Shing, 1992, J. Biol. Chem. 267:10931-10934; Risau, 1997, Nature 386:671-674). As in embryonic development, angiogenesis in the adult involves sprouting and growth of preexisting blood vessels, as well as remodeling (Risau, 1997, Nature 386:671-674).

Postnatally, angiogenesis has also been implicated in a number of diseases and disorders, in which abnormal endothelial growth may be either the primary cause of, or indirectly caused by, the pathological process. For example, abnormal endothelial cell proliferation has been postulated as playing a role in atherosclerosis. Further, the development of blood vessels in neoplasms plays a role in tumor growth and metastasis, and deregulated angiogenesis has been implicated in pathological processes such as rheumatoid arthritis, retinopathies, hemangiomas, and psoriasis (Folkman, 1995, Nature Med. 1: 27-31; Hanahan and Folkman, 1996, Cell 86:353364).

### 2.3 Molecular Mechanisms of Vasculogenesis and Angiogenesis

The genetic and molecular mechanisms that control development of the vascular system are beginning to be understood (reviewed in Folkman and D'Amore, 1996, Cell 87:1153-1155; Risau, 1997, Nature 386:671-674). Generation of angioblasts from paraxial and lateral plate mesoderm requires the function of members of the fibroblast growth factor family. Recent evidence suggests that vascular endothelial growth factor (“VEGF”) and its cellular receptors (VEGFR-1 or flt-1 and VEGFR-2 or flk-1) are essential for vasculogenesis during embryogenesis. VEGF, which is related to platelet-derived growth factor (“PDGF”), is secreted by the endoderm and functions in a paracrine manner (Breier et al., 1992, Development 114: 521-532; Breier and Risau, 1996, Trends Cell Biol. 6:454-456), whereas its tyrosine kinase receptors are co-expressed by the mesoderm-derived angioblasts and endothelial cells (Fong et al., 1995, Nature 376:66-70; Millauer et al., 1993, Cell 72:835-846; Yamaguchi et al., 1993, Development 118:489-498). Disruption of the gene for VEGFR-2 (flk-1) in mice results in a block to differentiation of angioblasts into endothelial cells and leads to embryonic death at day 8.5-9.5 (Shalaby et al., 1995, Nature 376:62-66). Disruption of the gene for the other receptor, VEGFR-1 (flt-1), leads to abnormal assembly of endothelial cells in blood vessels and embryonic death at day 9 of development (Fong et al., 1995, Nature 376: 66-70). Inactivation of a single VEGF allele leads to abnormal vascular structures and is lethal in the mouse embryo at about day 11 (Carmeliet et al., 1996, Nature 380:435-439; Ferrara et al., 1996, Nature 380:439-442). Therefore, each of these components of the VEGF signaling pathway is essential for vascular development and there is no functional redundancy among the ligand or its receptors. VEGF signal transduction involves receptor tyrosine kinase autophosphorylation, activation of phosphoinositol 3-kinase, phospholipase C γ and MAP kinase (Breier and Risau, 1996, Trends Cell Biol. 6:454-456). However, endothelial cell specific genes that lie downstream in the VEGF/flk-1flt-1 signaling pathway have not yet been conclusively identified.

Normal angiogenesis is thought to depend on a balance between inducers and inhibitors of endothelial cell proliferation and migration (Hanahan and Folkman, 1996, Cell 86:353-364). Positive regulatory molecules include VEGF and angiopoietin-1 (“Ag1”; Davis et al., 1996, Cell 87: 1161-1169). Ang-1 signals through the tyrosine kinase receptor Tie2/Tek that is specifically expressed on endothelial cells and early hematopoietic cells (Dumont et al., 1992, Oncogene 7:1471-1480). Absence of Ang1, its receptor Tie2/Tek, or Tie1, a closely related orphan receptor, causes severe abnormalities of the vascular system in both mouse and human. These findings suggest that these genes and their products play a role in angiogenesis and remodeling that occurs subsequent to VEGF and its receptors (Sato et al., 1995, Nature 376:7074; Suri et al., 1996, Cell 87:1171-1180; Vikkula et al., 1996, Cell 87:1181-1190). Recently, Angiopoietin-2 (“Ang2”), a factor closely related to Ang1, has been identified and shown to function as a natural antagonist for Ang1 and its Tie2 receptor (Maisonpierre et al., 1997, Science 277:55-60). Other inhibitors of angiogenesis include angiostatin, an internal fragment of plasminogen (O'Reilly et al., 1994, Cell 79:315-328) and endostatin, a C-terminal fragment of collagen XVIII (O'Reilly et al., 1997, Cell 88:277-285), both of which were isolated from tumors. However, their precise roles in normal angiogenesis and their relationship to the Ang1/Tie2 signaling pathway remain unknown.

### 2.4 Model Systems for Studying Endothelial Cell Differentiation

Several different model systems have been developed to study vasculogenesis and angiogenesis. The most common in vivo models are the chick embryo chorioallantoic membrane (“CAM”) and the rabbit retina. More recently, in vitro cultures of embryonic stem (“ES”) cell derived embryoid bodies have been used to study differentiation of angioblasts and the formation of vascular channels (Wang et al., 1992, Development 14:303-316). Finally, hemangioblastoma or yolk sac-derived endothelial cell lines have been established that form capillary tubes when grown in matrix-containing gels (Obeso et al., 1990, Lab. Invest. 63:259-269; Wang et al., 1996, Devel. Biol. Anim. 32:292-299).

### 2.5 The Human Gene DB 1

The present invention relates to the discovery of a novel mouse gene, Vezf1, expressed in association with angiogenic and vasculogenic events. Sequencing of the cDNA of Vezf1 revealed that it encoded a protein with over 98 percent homology to the previously identified human gene DB1 (Koyano-Nakagawa et al., 1994, Mol. Cell. Biol. 14:5099-5107). DB1 protein, a 516 amino acid protein with six zinc finger motifs, was found to be widely expressed in human blood cells and adult organs (Id.). It was identified in the Jurkat T cell line as a nuclear factor with sequence-specific DNA binding to a CT/GC-rich region of the promoter of the interleukin 3 (“IL-3”) gene (Id.). Any role for DB1 in endothelial cell development, angiogenesis, or vasculogenesis had not been known prior to the present invention. Rather, since no transcriptional activation of the IL-3 promoter could be detected, the ill vivo function of DB1 remained elusive.

### 3. Summary of The Invention

The present invention relates, at least in part, to the discovery of a novel mouse gene, referred to herein as Vezf1 (for “Vascular endothelial zinc finger 1”), which is expressed in association with vasculogenesis and angiogenesis.

In a first series of embodiments, the present invention provides for a nucleic acid encoding Vezf1, and a Vezf1 protein having an amino acid sequence as encoded by that nucleic acid.

In a second set of embodiments, the present invention provides for a method of identifying an endothelial cell, comprising identifying the expression of a Vezf1-encoding RNA or a Vezf1 protein in the cell.

In a third series of embodiments, the present invention provides for a method of diagnosing a vascular disorder in a subject, whereby the presence, amount, and/or molecular characteristics of the Vezf1 gene, its corresponding mRNA or a cDNA thereof, or its product protein arc determined and compared to normal values. Differences in the molecular characteristics of the Vezf1 gene or its expression would support a diagnosis of a vascular disorder. In a related set of embodiments, subjects carrying a defective Vezf1 gene may be identified according to the invention; such individuals may be at risk of developing a vascular disorder or passing such a condition to their progeny.

In a fourth set of embodiments, the present invention provides for a non-human animal model system for studying angiogenesis and vasculogenesis, and in particular, the role of Vezf1 in these processes. Such models may be used to study the effects of systemic or local increases in Vezf1 expression, or, alternatively, decreased or aberrant Vezf1 expression. Such model systems, or cell lines derived therefrom, may be used to identify agents that act as agonists or antagonists of Vezf1 activity.

In a fifth set of embodiments, the present invention provides for methods of treating vascular diseases and disorders. For example, but not by way of limitation, the inhibition of Vezf1 expression may be used to inhibit angiogenesis, for example, in a neoplasm. Alternatively, increased expression of Vezf1 may be useful in promoting wound healing or in treating clinical disorders related to ischemia.

### 4. Description of The Figures

FIG. 1. Nucleic acid sequence of mouse Vezf1 cDNA (SEQ ID NO: 1) and deduced amino acid sequence (SEQ ID NO: 2).

FIG. 2. Nucleic acid sequence of human DB1 cDNA (SEQ ID NO: 3) and deduced amino acid sequence (SEQ ID NO: 4) (Koyano-Nakagawa et al., 1994, Mol. Cell. Biol. 14:5099-5107; GeneBank Acc. No. D28118).

FIG. 3. Schematic overview of a genetic screen for developmentally regulated genes using retroviral entrapment vectors with human alkaline phosphatase (“AP”) as a reporter gene.

FIG. 4A-X. Differential AP reporter gene expression during in vitro differentiation of ES cell clones with entrapment vector insertions. ES cells from individual clones were induced to spontaneously differentiate into embryoid bodies. Aliquots from day-4, day-7, day-10 embryoid bodies, or terminally differentiated cultures, respectively, were stained for AP activity with BCIP+NBT (A-L) or BM purple (Boehringer Mannheim) (M-X), and photographed using a Nikon SMZ-U stereomicroscope, as follows:

(A,B): day-4,day-7 embryoid bodies originating from uninfected ES cells;

(C,D): day-4, day-10 embryoid bodies originating from PT-AP clone IV E3-8;

(E,F): day-4, day-10 embryoid bodies originating from PT-AP clone 7-65;

(G,H): day-4, day-7 embryoid bodies originating from PT-IRES-AP clone 1-13;

(I,J): day-7, day-10 embryoid bodies originating from PT-IRES-AP clone 1-58;

(K,L): day-7, day-10 embryoid bodies originating from PT-IRES-AP clone 2-86;

(M,N): day-7, day-10 embryoid bodies originating from PT-IRES-AP clone 2-51;

(O,P): day-7, day-10 embryoid bodies originating from PT-IRES-AP clone 4-45;

(Q,R): day-7, day-10 embryoid bodies originating from PT-IRES-AP clone 6-33;

(S,T): day-4, day-10 embryoid bodies originating from PT-IRES-AP clone 7-26;

(U,V): day-7, re-plated embryoid bodies originating from PT-IRES-AP clone 9-E5; and

(W,X): day-7, day-10 embryoid bodies originating from PT-IRES-AP clone 9-G7.

FIG. 5 A-D. AP expression in ES cells of the #1-13 clone. (A) Day-4 embryoid bodies in suspension culture. (B) Day-7 embryoid bodies in suspension culture. (C) Day-10 embryoid bodies in suspension culture. (D) Re-plated day-4 embryoid body monolayers in gelatin-coated culture dishes.

FIG. 6A-B. (A) Retroviral vector insertion #1-13 with flanking genomic DNA. (B) Southern analysis, using a 0.7 kb genomic DNA fragment 3′ of the insertion's EcoRI site as a probe, of high molecular weight DNA from R1 ES cells, cells of the #1-13 insertion-bearing ES cell clone, and transgenic mice homozygous (−/−) or heterozygous (+/−) for the #1-13 insertion. DNA from wild-type CD-1 (+/+) mice lacking the #1-13 mutation was used as a control, and λ DNA digested with EcoRI and BamHI was used as a size marker (left lane).

FIG. 7. Amino acid sequence alignment of residues 168-360 of mouse Vezf1 (SEQ ID NO: 12) and residues 275-469 of mouse Pur-1 (SEQ ID NO: 13).

FIG. 8. Retroviral insertion in the 3′ untranslated region of Vezf1 in ES clone #1-13. Probes A, B and C correspond to cDNA clones of Vezf1, probe A corresponding to nucleotides 1-776, probe B corresponding to nucleotides 97-1444, and probe C corresponding to nucleotides 1397-2820.

FIG. 9(A-F). Expression of AP in developing embryos containing the #1-13 retroviral insertion, as analyzed by histochemical staining with BM purple (Boehringer Mannheim). Developmental stages studied were (A) E7.25, (B) E7.5, (C) E8.5, (D) E9.5, (E) E10.5 and (F) E11.5.

FIG. 10(A-H). Expression of AP in histological sections of developing embryos containing the #1-13 retroviral insertion, as measured by histochemical staining. Developmental stages studied were (A) E7.25 (14X magnification), (B) E7.25 (100X magnification), (C) E7.25 (50X magnification), (D) E7.25 (200X magnification), (E) E7.5 (50X magnification), (F) E7.5 (100X magnification), (G) E8.0 (50X magnification) and (H) E8.0 (200X magnification).

FIG. 11A-H. Expression of AP in histological sections of embryos containing the #1-13 retroviral insertion at stages E8.5 (A-D) and E9.5 (E-H) measured by histochemical staining, showing expression in (A and E) the whole embryo, (B) the head fold, (C) the heart, (D and H) the yolk sac, (F) the neural tube; and (G) the cardiac ventricle.

FIG. 12 (A-J) Expression of Vezf1 mRNA in developing embryos as measured by in situ hybridization. Developmental stages and tissues evaluated were (A) E7.25 (dark field), (B) E7.25 (bright field), (C) E8.5 (dark field), (D) E8.5 (bright field), (E) E9.5 (dark field), (F) dorsal aorta (dark field), (G) somites (dark field), (H) heart (dark field), (I) lung (bright field) and (J) kidney (bright field).

FIG. 13A-C. Northern blot analysis of Vezf1 RNA expression in (A) various stages of embryoid bodies, ES cells, and mouse embryos; (B) in hematopoietic and endothelial cell lines; and (C) in various tissues of adult mice.

FIG. 14A-B. Southern analysis of hybridization of Vezf1 probe with DNAs from various species restricted with either BamHI (lanes marked “B”), EcoRI (lanes marked “R”), or HindIII (lanes marked “H”).

FIG. 15A-B. Map of Jackson BSS chromosome 2 showing location of the Vezf1 gene. A DNA panel from the Jackson Laboratory community resource (C57BL/6J×M. spretus), BSS interspecific backcross, was used to map the Vezf1 locus using PCR techniques. (A) Haplotype figure from the Jackson backcross showing part of Chromosome 2 with loci linked to Vezf1 (black boxes: C57BL6/JEi allele). (B) Map figure from the Jackson BSS backcross showing the proximal end of Chromosome 2. The map is depicted with the centromere toward the top. Raw data from the Jackson Laboratory were obtained from the World Wide Web address http://www.jax.org/resources/documents/cmdata.

FIG. 16. Restriction enzyme map of 20 kb mouse genomic fragment containing the promoter element and first two exons.

FIG. 17. Structure of plasmid vectors expressing wild type and mutant Vezf1. The open box (top) depicts the Vezf1 cDNA from nucleotides 1 to 2280 (ATG transcriptional start at nucleotide 39) with coding regions for six putative zinc fingers (indicated by black bars). DZF- Vezf1 contains a deletion from nucleotide 309 to 992, including zinc fingers 2 to 6 and half of the first zinc finger. Vezf1-ZF contains nucleotides 309 to 992 of the cDNA including zinc fingers 2 to 6 and half of the first zinc finger.

FIG. 18. Retroviral entrapment vectors. The depicted constructs contain a 1.9 kb fragment of the human placental alkaline phosphatase gene (AP) as a reporter gene.GT-AP contains the Mo-MLV env splice acceptor site (SA) and PT-IRES-AP contains a 0.6 kb IRES fragment from encephalomyocarditis virus (EMCV). All constructs contain a 1.3 kb neo expression cassette with a PGK promoter and the neo^{r} gene. The transcriptional orientation of the AP gene and the neo expression cassette are indicated by arrows. The vectors contain 5′ Mo-MLV sequences up to nucleotide 1560 derived from pMov9, including the 5′LTR, the packaging site Ψ, and 1 kb of gag sequences. The 3′ end of the vectors contain Mo-MLV sequences from nucleotide 7674, including the 3′ LTR with the 200 bp E. coli sup F gene inserted at position 7911 in U3, derived from pLTR^{sup-1}. A 294 bp deletion was generated in pLTR^{sup-1} from PvuII (position 7935) to SacI (position 8229) containing the retroviral enhancer and promoter sequences. The relative position of the supF insert, the deletion of enhancer and promoter (enh/pro⁻) and the polyadenylation [p(A)] sequence are indicated. The length of different fragments and the relevant restriction sites used for the construction of the vectors are shown. E=EcoRI; K=KpnI; X=XhoI.

### 5. DETAILED DESCRIPTION OF THE INVENTION

For purposes of clarity, and not by way of limitation, the detailed description of the invention is divided into the following subsections:

(i) the Vezf1 gene and its products;

(ii) diagnostic methods;

(iii) model systems; and

(iv) methods of treatment.

### 5.1 The Vezf1 Gene and its Products

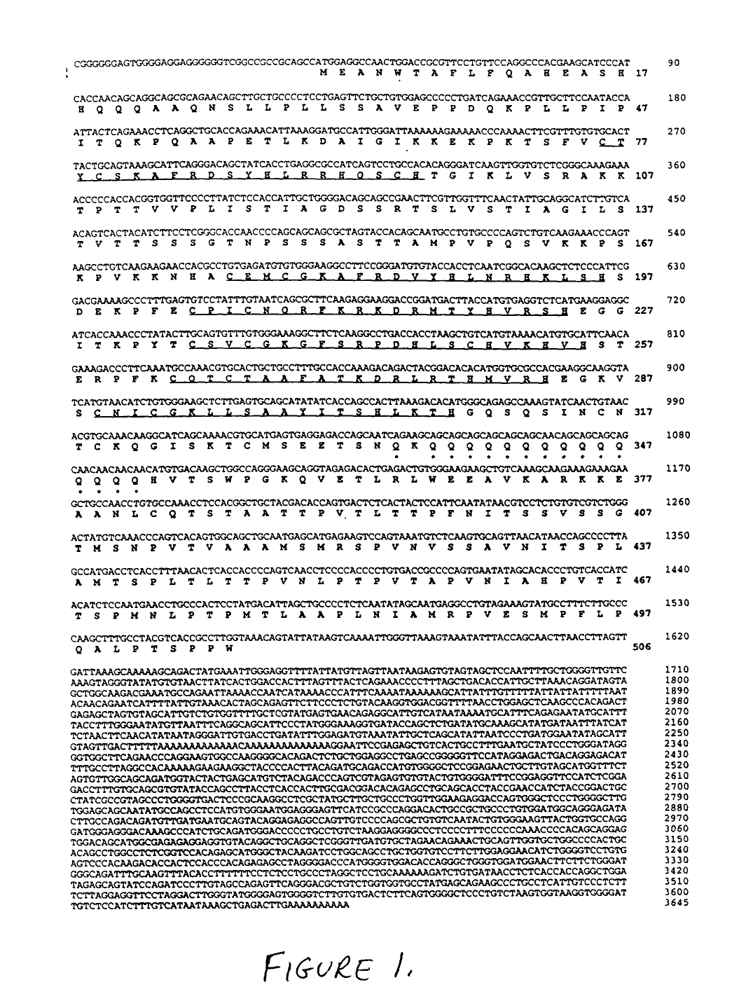

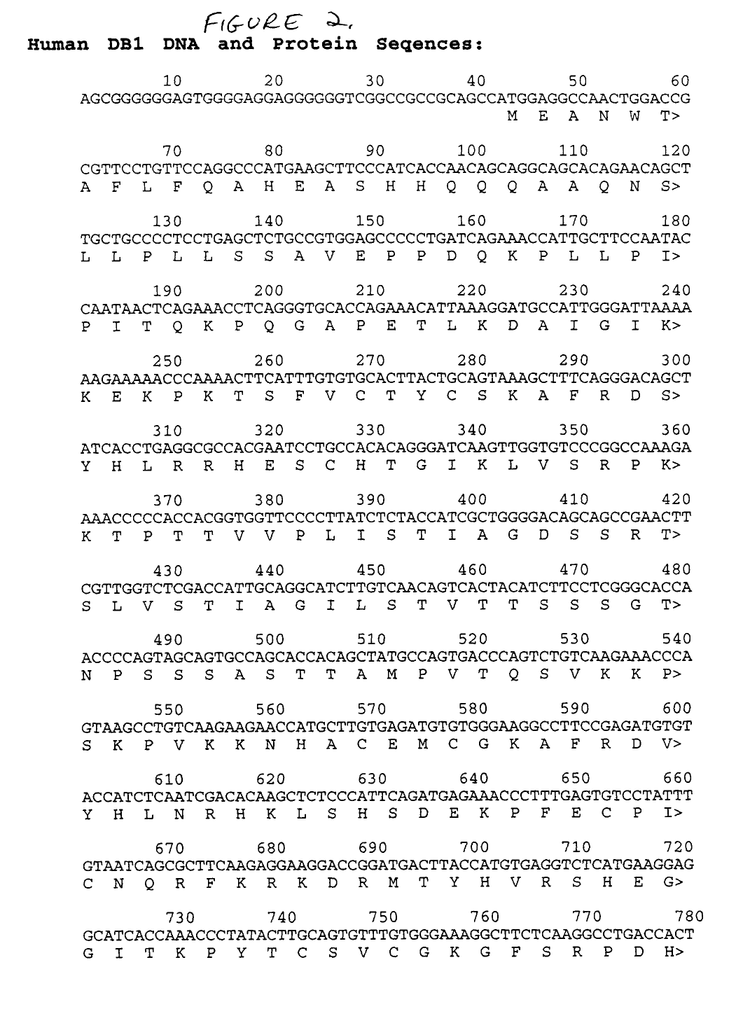

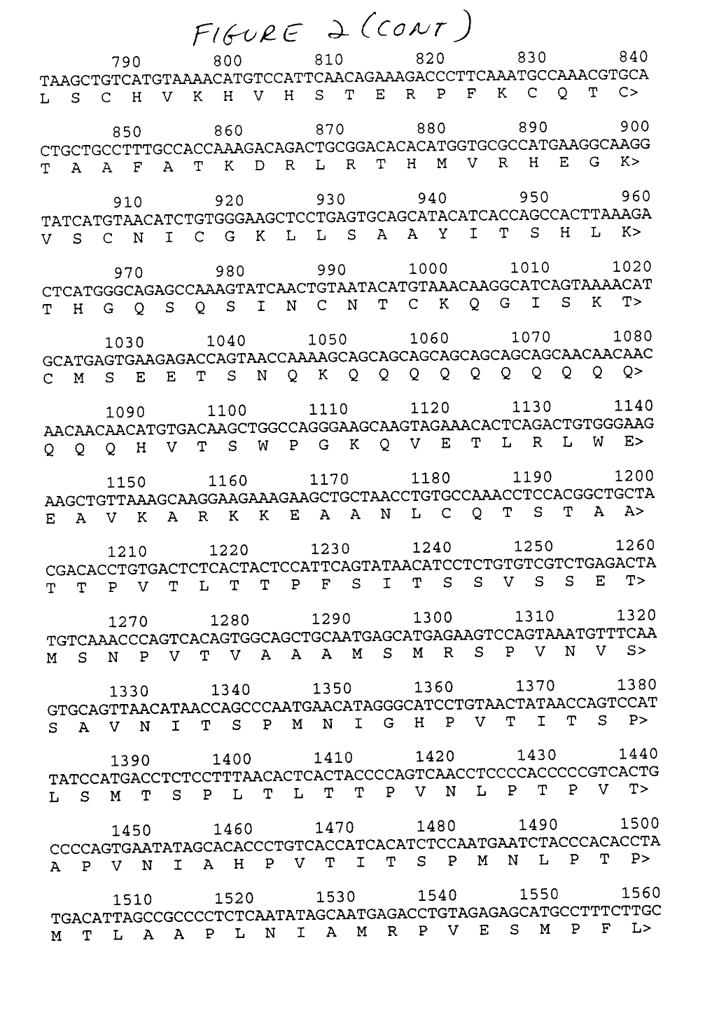

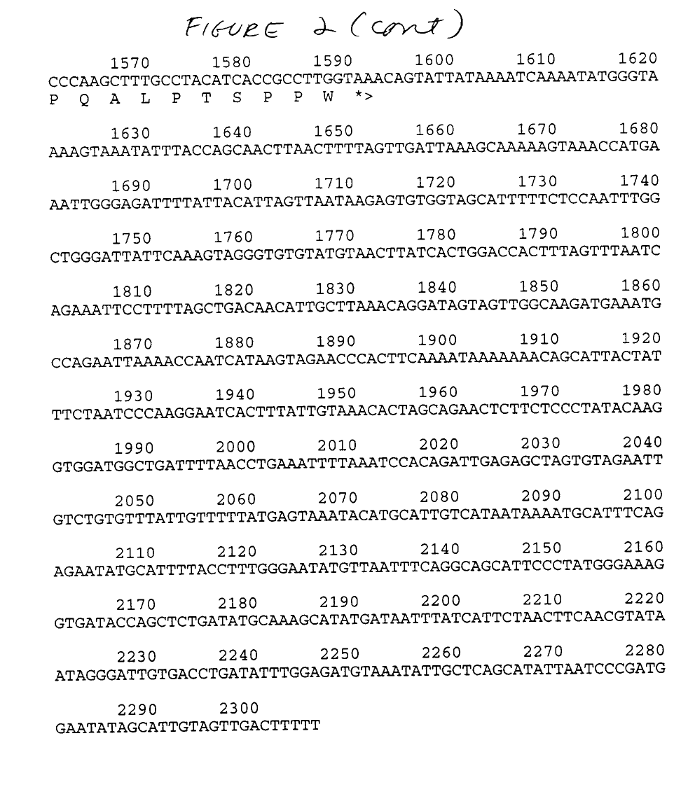

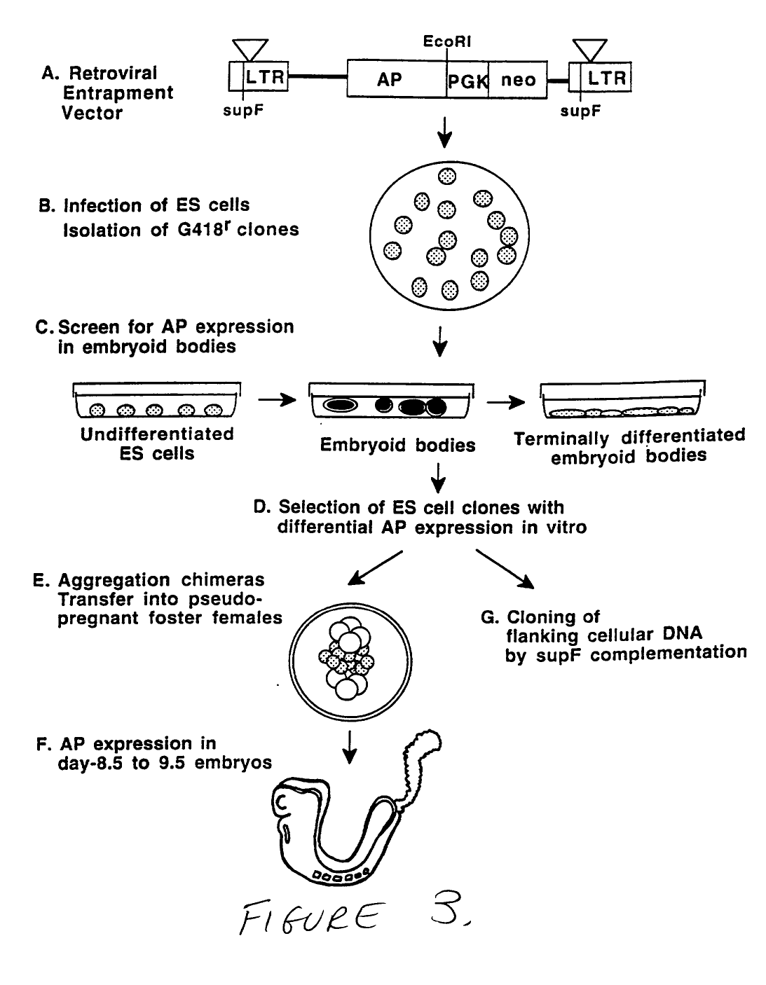

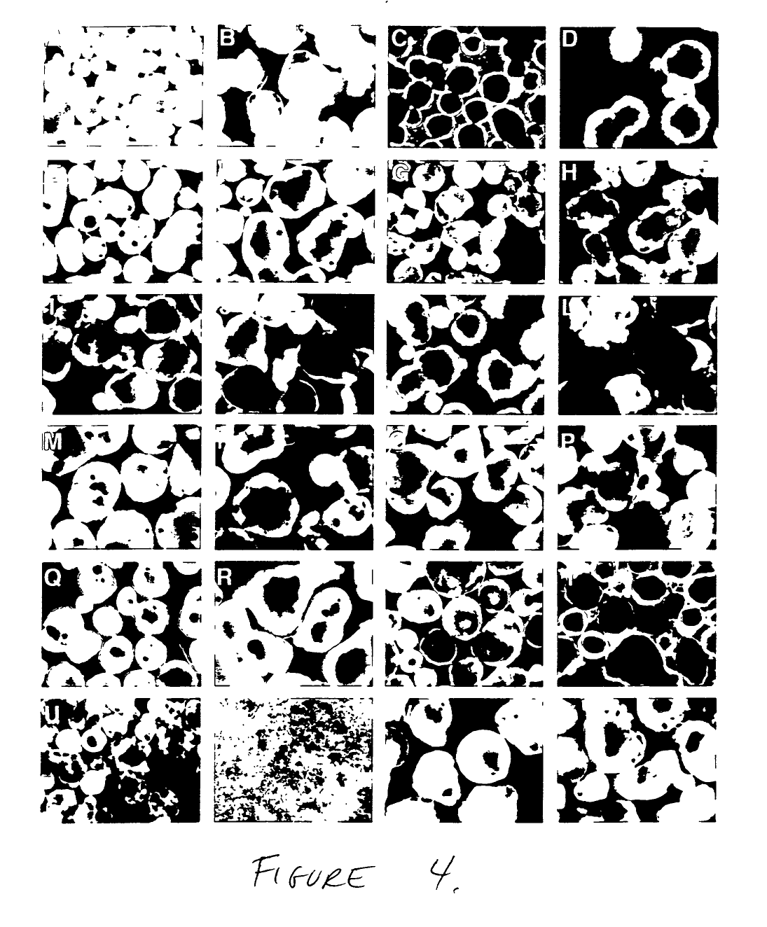

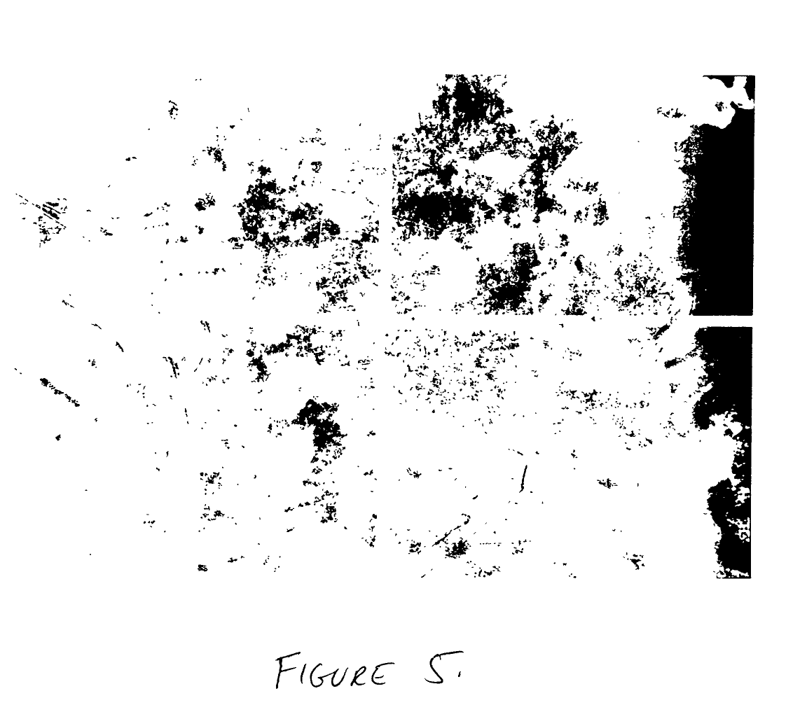

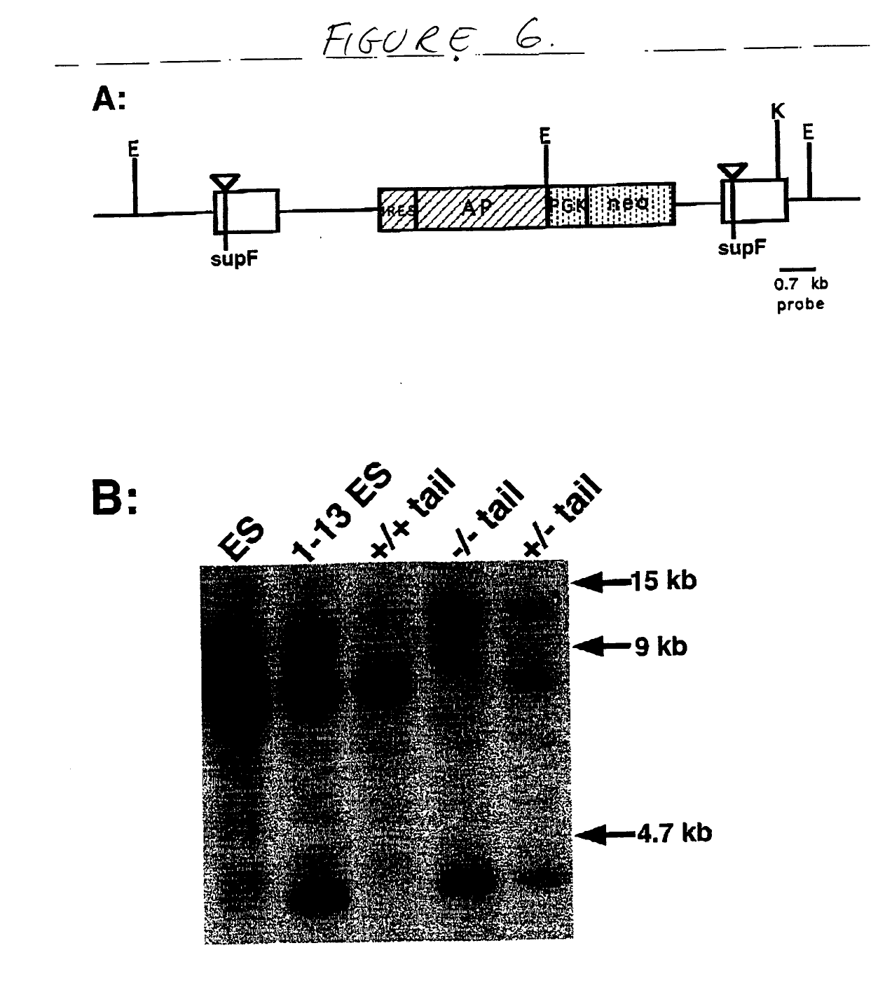

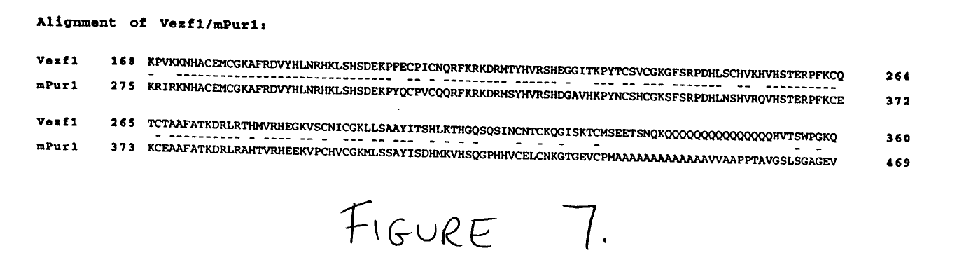

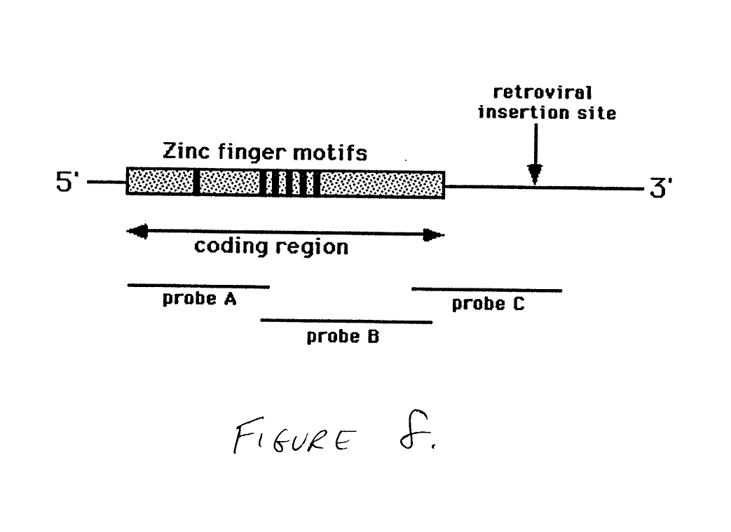

The present invention relates to a Vezf1 gene (including its control elements), RNA transcribed from the Vezf1 gene or any cDNA or antisense counterparts thereof, and its encoded protein. The term “Vezf1 gene”, as used herein, collectively includes the mouse Vezf1 gene, its human counterpart (also referred to as DB1), and homologs thereof in other species which are at least about 90 percent, and preferably about 95 percent, homologous to the mouse Vezf1 gene (homology determined by MACVECTOR, Blast Search Algorithm (Netscape Navigator, 3.01), and/or which hybridize to the mouse Vezf1 gene under stringent hybridization and wash conditions, such as hybridization in 50 percent formamide, 5×SSPE, 2×Denhardt's, 0.5 percent SDS, 100 μg/ml salmon sperm DNA, at 37° C.; and washes in 0.2×SSC, 0.1 percent SDS at 50° C. The term “Vezf1 cDNA”, as used herein, collectively includes the mouse Vezf1 cDNA having a sequence as set forth in FIG. 1 (SEQ ID NO: 1), its human counterpart (referred to herein as DB1), having a nucleic acid sequence as set forth in FIG. 2 (SEQ ID NO: 3) and homologs thereof in other species which are at least about 90 percent, and preferably about 95 percent, homologous to mouse Vezf1 cDNA (as determined by MACVECTOR, see above) and/or which hybridize to the mouse Vezf1 gene under stringent hybridization and wash conditions as set forth above.

The present invention further provides for a Vezf1 promoter sequence, as contained in a 20 kb genomic fragment (FIG. 16), isolated by screening a 129 mouse genomic lambda phage library. The genomic fragment contains approximately 15 kb of genomic sequences upstream of the coding ATG, the first and second exons and the first intron of the Vezf1 gene. The promoter sequence contained in this fragment may be identified using standard techniques. The present invention further provides for the human Vezf1 promoter, which may be identified by hybridization to the foregoing mouse 20 kb genomic fragment (as contained in Vezf1.N, see below) under stringent hybridization and wash conditions (hybridization in 50 percent formamide, 5×SSPE, 2×Denhardt's, 0.5 percent SDS, 100μ/ml salmon sperm DNA, at 37° C.; and washes in 0.2×SSC, 0.1 percent SDS at 50° C.).

Two purified and isolated nucleic acids, together comprising Vezf1 cDNA, termed mVezf1.1 (3-1444) and mVezf1.2 (1394-2820), containing nucleic acid residues 3-1444 (in a 1.444 kb EcoRI-EcoRI cDNA fragment) and 1394-2820 (in a 1.429 kb EcoRI-EcoRI cDNA fragment) of murine Vezf1 cDNA, respectively, in pBluescript II SK (grown in DH5α) and a nucleic acid, termed m Vezf1.N, containing the forementioned 20 kb genomic NotI-NotI fragment in pBluescript II SK (grown in DH5α), have been deposited under the terms of the Budapest Treaty with the American Type Culture Collection (ATCC), having an address of 12301 Parklawn Drive, Rockville, Md., 20852 on ______, 1998 and assigned accession nos. ______, ______, and ______, respectively.

The present invention also provides for nucleic acid molecules which hybridize to a Vezf1 gene, including complementary DNA and RNA molecules (referred to as “corresponding” to the Vezf1 gene), including mRNA transcripts of the Vezf1 gene as well as nucleic acid molecules which are “anti-sense”, that is to say, complementary to a mRNA transcript of the Vezf1 gene. Preferably, the conditions for hybridization and washing are as follows: hybridization in 50 percent formamide, 5×SSC, 2×Denhardt's, 0.5 percent SDS, 100 μg/ml salmon sperm DNA at 42° C., and washes is 0.2 SSC, 0.1 percent SDS at 50° C.

The present invention still further provides for purified and isolated Vezf1 proteins. The term “Vezf1 protein”, as used herein, includes murine Vezf1 protein, having an amino acid sequence as set forth in FIG. 1 (SEQ ID NO: 2), the human DB1 protein having an amino acid sequence as set forth in FIG. 2 (SEQ ID NO: 4), and proteins which are at least 90 percent, and preferably 95 percent, homologous to mouse Vezf1 protein as determined by MACVECTOR and the Blast Search Algorithm.

The Vezf1 gene or a corresponding cDNA or RNA may be incorporated into any suitable cloning or expression vector, operably linked to appropriate control elements (e.g., promoter/enhancer elements, ribosomal binding sites, polyadenylation sites, termination sites, etc.). Examples of such vectors include, but are not limited to, herpes simplex viral based vectors such as pHSV1 (Geller et al., 1990, Proc. Natl. Acad. Sci. U.S.A. 87:8950-8954); retroviral vectors such as MFG (Jaffee et al., 1993, Cancer Res. 53:2221-2226), and in particular Moloney retroviral vectors such as LN, LNSX, LNCX, LXSN (Miller and Rosman, 1989, Biotechniques 7:980-989); vaccinia viral vectors such as MVA (Sutter and Moss, 1992, Proc. Natl. Acad. Sci. U.S.A. 89:10847-10851); adenovirus vectors such as pJM17 (Ali et al., 1994, Gene Therapy 1:367-384; Berker, 1988, Biotechniques 6:616-624; Wand and Finer, 1996, Nature Medicine 2:714-716); adeno-associated virus vectors such as AAV/neo (Mura-Cacho et al., 1992, J. Immunother. 11:231-237); lentivirus vectors (Zufferey et al., 1997, Nature Biotechnology 15:871-875); and plasmid vectors such as pCDNA3 and pCDNA1 (InVitrogen), pET 11a, pET3a, pET11d, pET3d, pET22d, pET12a and pET28a (Novagen); plasmid AH5 (which contains the SV40 origin and the adenovirus major late promoter), pRC/CMV (InVitrogen), pCMU II (Paabo et al., 1986, EMBO J. 5:1921-1927), pZipNeo SV (Cepko et al., 1984, Cell 37:1053-1062), pSRα(DNAX, Palo Alto, Calif.) and pBK-CMV, pSPTg.T2FpAXK and pSPTg.2FXK (Schaleger et al., 1997, Proc. Natl. Acad. Sci. U.S.A. 94:3058-3063).

In specific, nonlimiting embodiments of the invention, a Vezf1 gene or a corresponding cDNA or RNA may be incorporated into an expression vector as part of a dicistronic expression cassette together with a second gene. For example, such a gene may be a reporter gene (e.g., human placental alkaline phosphatase, luciferase, β-galactosidase, etc.).

In other specific, nonlimiting embodiments of the invention, an expression vector may comprise a Vezf1 gene (or a corresponding cDNA or RNA) operably linked to a heterologous promoter (i.e., a promoter not found, in nature, linked to the Vezf1 gene) known or believed to be selectively expressed in vascular endothelial cells, including, but not limited to, the Tie2 promoter enhancer (Schlaeger et al., 1997, Proc Natl. Acad. Sci. U.S.A. 94:3058-3063) and the flk-1 promoter/enhancer (Rönicke et al., 1996, Circ. Res. 79:277-285). In alternative embodiments, the heterologous promoter may be an inducible promoter, such as the metallothionine promoter.

The present invention further provides for antibody molecules which specifically bind to a Vezf1 protein. According to the invention, a Vezf1 protein, its fragments or other derivatives, or analogs thereof, may be used as an immunogen to generate antibodies which immununospecifically bind such an immunogen. Such antibodies include but are not limited to polyclonal, monoclonal, chimeric, single chain, Fab fragments, and an Fab expression library. In specific embodiments, antibodies to mouse Vezf1 protein or to human DB-1 protein are produced.

Various procedures known in the art may be used for the production of polyclonal antibodies which specifically bind to a Vezf1 protein. In a particular embodiment, rabbit polyclonal antibodies to an epitope of a Vezf1 protein having an amino acid sequence set forth in FIG. 1 (SEQ ID NO: 2) or FIG. 2 (SEQ ID NO: 4) may be obtained. For the production of antibody, various host animals can be immunized by injection with the native Vezf1 protein, or a synthetic version, or derivative (e.g., fragment) thereof, including but not limited to rabbits, mice, rats, etc. Various adjuvants may be used to increase the immunological response, depending on the host species, and including but not limited to Freund's (complete or incomplete) adjuvant, mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum.

For preparation of monoclonal antibodies directed toward a Vezf1 protein, any technique which provides for the production of antibody molecules by continuous cell lines in culture may be used. Examples of such techniques include the hybridoma technique originally developed by Kohler and Milstein (1975, Nature 256:495-497), as well as the trioma technique, the human B-cell hybridoma technique (Kozbor et al., 1983, Immunology Today 4:72), and the EBVhybridoma technique to produce human monoclonal antibodies (Cole et al., 1985, in Monoclonal Antibodies and Cancer Therapy, Alan R. Liss, Inc., pp. 77-96). In an additional embodiment of the invention, monoclonal antibodies can be produced in germ-free animals utilizing recent technology (PCT/US90/02545). According to the invention, human antibodies may be used and can be obtained by using human hybridomas (Cote et al., 1983, Proc. Natl. Acad. Sci. U.S.A. 80:2026-2030) or by transforming human B cells with EBV virus in vitro (Cole et al., 1985, in Monoclonal Antibodies and Cancer Therapy, Alan R. Liss, pp. 77-96). In fact, according to the invention, techniques developed for the production of “chimeric antibodies” (Morrison et al., 1984, Proc. Natl. Acad. Sci. U.S.A. 81:6851-6855; Neuberger et al., 1984, Nature 312:604-608; Takeda et al., 1985, Nature 314:452-454) by splicing the genes from a mouse antibody molecule specific for Vezf1 together with genes from a human antibody molecule of appropriate biological activity can be used; such antibodies are within the scope of this invention.

According to the invention, techniques described for the production of single chain antibodies (U.S. Pat. No. 4,946,778) can be adapted to produce Verf1-specific single chain antibodies. An additional embodiment of the invention utilizes the techniques described for the construction of Fab expression libraries (Huse et al., 1989, Science 246:12751281) to allow rapid and easy identification of monoclonal Fab fragments with the desired specificity for Vezf-1 protein derivatives, or analogs.

Antibody fragments which contain the idiotype of the molecule can be generated by known techniques. For example, such fragments include but are not limited to: the F(ab1), fragment which can be produced by pepsin digestion of the antibody molecule; the Fab′ fragments which can be generated by reducing the disulfide bridges of the F(ab1), fragment, the Fab fragments which can be generated by treating the antibody molecule with papain and a reducing agent, and Fv fragments.

In the production of antibodies, screening for the desired antibody can be accomplished by techniques known in the art, e.g. ELISA (enzyme-linked immunosorbent assay). For example, to select antibodies which recognize a specific domain of a Vezf1 protein, one may assay generated hybridomas for a product which binds to a Vezf1 fragment containing such domain. For selection of an antibody that specifically binds a first Vezf1 homolog but which does not specifically bind a different Vezf1 homolog, one can select on the basis of positive binding to the first Vezf1 homolog and a lack of binding to the second Vezf1 homolog.

### 5.2 Diagnostic Methods

The present invention provides for a method of diagnosing a vascular disorder in a subject, whereby the presence, amount, and/or molecular characteristics of the Vezf1 gene, its corresponding mRNA or a cDNA thereof, or its gene product are determined and compared to normal values. Differences in the molecular characteristics of the Veftz1 gene or its expression would support a diagnosis of a vascular disorder or would indicate that the subject is capable of genetically transmitting such a disorder to its progeny.

The term “vascular disorder”, as used herein, includes disorders which primarily arise in the vasculature as well as conditions which primarily involve other tissues or organs but in which angiogenesis is either undesirable (e.g., neoplasms and rheumatoid arthritis) or advantageous (e.g., in ischemic conditions and wound healing).

In particular, the present invention provides for a method of diagnosing a vascular disorder in a subject, comprising measuring the amount of a Vezf1 gene product, where the gene product is selected from the group consisting of RNA and protein, in a test sample taken from the subject and comparing that amount to the amount of Vezf1 gene product in a matched control sample, wherein a difference in the amount of Vezf1 gene product in the test sample and the control sample correlates with the presence of a vascular disorder in the subject.

In particular, the present invention provides for a method of diagnosing a disorder involving angiogenesis in a subject, comprising measuring the amount of a Vezf1 gene product, where the gene product is selected from the group consisting of RNA and protein, in a test sample taken from the subject and comparing that amount to the amount of Vezf1 gene product in a matched control sample, wherein a difference in the amount of Vezf1 gene product in the test sample and the control sample correlates with the presence of a disorder involving angiogenesis in the subject. An increased amount of Vezf1 gene product in the test sample positively correlates with increased angiogenesis in the disorder, and a decreased amount of Vezf1 gene product in the test sample positively correlates with decreased angiogenesis in the disorder.

The present invention also provides for a method of diagnosing a heritable vascular disorder in a subject (including the ability to have an offspring suffering from a vascular disorder which is not apparent in the parental phenotype), comprising characterizing a Vezf1 gene in the subject and comparing the characteristics of the gene to the normal Vezf1 gene, where a difference in characteristics of the Vezf1 gene in the subject and the normal Vezf1 gene correlates with the presence of a heritable vascular disorder in the subject.

For example, the characteristics of a Vezf1 gene in a subject may be evaluated by analyzing the DNA of the subject. For example, a sample of DNA obtained from the subject may be prepared and then subjected to Southern analysis, using, as a hybridization probe, a Vezf1 gene or cDNA or portion thereof (hereafter, “a Vezf1 probe”). Using such a technique, the DNA of the subject may be cleaved by one or more restriction endonucleases, the resulting fragments may be resolved electrophoretically, e.g. in an agarose gel, and then transferred to a membrane (e.g. nitrocellulose) and hybridized to a Vezf1 probe. A control DNA sample, subjected to the same treatments, may be run in parallel as a control. A different pattern of restriction fragments in the DNA sample of the subject relative to the pattern observed in control DNA may indicate an abnormality in the Vezf1 gene in the subject.

In specific, nonlimiting examples, the Verf1 probe may be a nucleic acid having a sequence as set forth in FIG. 1 (SEQ ID NO: 1) or FIG. 2 (SEQ ID NO: 3), or a subsequence thereof, wherein the subsequence is at least 10 nucleotides long, and more preferably at least 50 nucleotides long. The Vezf1 probe may be detectably labeled, for example, with a radioisotope or a detectable molecule, such as biotin.

Similarly, the expression of Vezf1 RNA in a subject may be evaluated. The size and amount of the Vezf1 mRNA transcript in a cell or tissue sample of a subject may be determined, for example, using Northern analysis, wherein RNA prepared from the sample is electrophoretically separated in the presence of formamide, transferred to a membrane (e.g. nitrocellulose), and then hybridized to a Vezf1 probe as described above. Where the size or amount of Vezf1 RNA in the sample differs relative to the size or amount of Vezf1 RNA in a matched control sample, a disorder in Vezf1 expression, or a disorder associated with abnormal Vezf1 expression, is indicated. In humans, the normal size of the Vezf1 (also known as DB-1) mRNA transcript is appoximately between 4.0 and 4.8 kb.

Further, the expression of Vezf1 protein in a subject may be evaluated. The size and amount of Vezf1 protein in a cell or tissue sample of a subject may be determined, for example, by Western blot analysis, wherein protein prepared from the sample is electrophoretically separated (e.g., in a polyacrylamide gel), transferred to a membrane (e.g., nitrocellulose) and then bound to anti- Vezf1 antibody. To quantitate the amount of protein present, an ELISA assay may be used. Where the size or amount of Vezf1 protein in the sample differs relative to the size or amount of Vezf1 protein in a matched control sample, a disorder in Vezf1 expression, or a disorder associated with abnormal Vezf1 expression, is indicated. The apparent normal size of the human Vezf-1 protein, DB-1, is about 56-80 kDa..

Still further, Vezf1 expression at the RNA or protein level may be used to detect or measure the presence or amount of vascular endothelial cells in a cell or tissue sample of a subject.

The detection of primary genetic defects in the Vezf1 gene (as indicated, for example, by restriction fragment length polymorphism or an abnormal size of mRNA transcript) or abnormalities in the level or timing of Vezf1 expression bear a positive correlation with a diagnosis of a heritable vascular disorder. The propensity of such a subject to transmit such a disorder to its offspring would be supported.

Where the Vezf1 gene itself in a subject is normal, and the control of its expression is normal, an abnormally high level of Vezf1 mRNA or protein may indicate the presence of a disease or disorder which increases proliferation of vascular endothelial cells. Examples of such conditions include, but are not limited to, damage to the existing vascular endothelium by trauma (including intrinsic and iatrogenic) or a disease process, such as atherosclerosis, neoplasia, or the generation of new tissue, as in wound healing or pannus formation in rheumatoid arthritis, or other disorders which result in the proliferation of blood vessels, such as malignant endothelial angiomatosis. As the metastatic potential of a malignant tumor may correlate with its vascularity, a determination of the level of Vezf1 mRNA or protein expression in a tumor sample may have prognostic value, where high levels may bear a positive correlation with increased metastatic potential.

Analogously, Vezf1 levels may be decreased in necrotic cells or tissues, post-mitotic or resting endothelial cells, such that a determination that the Vezf1 level in a tissue or cell is decreased relative to normal tissues may indicate that the cell or tissue is necrotic or in a resting state. Further, a decrease in Vezf1 level in a subject may indicate a regression in a condition associated with an increase in Vezf1 expression; as an example, the level of Vezf1 may fall in a tumor when the tumor regresses.

### 5.3 Model Systems

The present invention provides for non-human animal model systems which may be used to evaluate the effects of increasing, decreasing, or altering the temporal expression pattern of Vezf1 expression. Such animals may carry, as a transgene, an exogenously introduced Vezf1 gene or a Vezf1 cDNA, in some or all of their cells, and/or may have a structural alteration in one or more of their endogenous Vezf1 genes. Such an alteration may be a “knock-out” of one or more endogenous Vezf1 gene. The exogenous Vezf1 gene or cDNA may be present in some or all cells of the animal.

In specific, nonlimting embodiments of the invention, a Vezf1 cDNA exogenously introduced into a non-human animal may have a nucleic acid sequence as set forth in FIG. 1 (SEQ ID NO: 1) or FIG. 2 (SEQ ID NO: 3), and may be operably linked to a promoter element. The promoter element may be a Vezf1 promoter, another promoter selectively active in vascular endothelial cells (such as the Tie2 promoter), a promoter active during substantially the same developmental stages as the Vezf1 promoter, or an inducible promoter (such as the metallothionine or tetracycline promoter) or a promoter which directs widespread or ubiquitous expression (such as a human cytomegalovirus promoter or a retroviral LTR promoter).

Transgenic animals carrying a Vezf1 gene, or a portion or mutation thereof, may be created by standard techniques, including, but not limited to, techniques described in U.S. Pat. No. 4,736,866, PCT publication W082/04443, PCT publication W088/00239. Such animals may also be produced by infection with vectors carrying Vezf1 encoding nucleic acid in expressible form, or by inoculation with naked DNA encoding a Vezf1 protein or mutant or subfragment thereof. Animals in which the endogenous Vezf1 gene or its control elements have been altered by homologous recombination may be produced using techniques as set forth in U.S. Pat. No. 5,464,764 or Bradley, 1991, Current Op. Biotechnol. 2:823-829. The present invention also contemplates animals in which one or more endogenous gene has been “knocked out” and an exogenous Vezf1 gene or cDNA has been introduced.

Non-limiting examples of animals which may serve as non-human animal model systems include mice, rats, rabbits, chickens, sheep, goats, cows, pigs, and non-human primates.

For example, non-human animals may be produced in which extra copies of the Vezf1 gene or cDNA have been introduced, or a Vezf1 gene under a strong promoter has been created or introduced into the animal, such that the level of Vezf1 protein in the animal, or in a subset of cells of the animal, has been increased relative to normal levels. Preferably the increase is by at least 25 percent. Such animals may be used to study the effects of increased Vezf1 levels, or may be used to identify compounds which act as antagonists of Vezf1 activity.

Alternatively, non-human animals may be produced which have lower than normal levels of Vezf1 expression in some or all cells, by virtue of a defect introduced in one or more alleles of the endogenous Vezf1 gene and/or its control elements. For example, but not by way of limitation, one or both alleles of the endogenous Vezf1 gene in the animal may be eliminated or mutated by gene targeting techniques. Alternatively, an additional Vezf1 gene with altered structure may be introduced which competes with endogenous Vezf1 and thereby inhibits native Vezf1 activity. Examples of mutated forms of the Vezf1 gene include variant genes which produce a truncated Vezf1 protein, for example,a Vezf1 protein lacking one or more zinc finger motifs, or a Vezf1 protein which eliminates non-zinc-finger related amino acid sequence (see, for nonlimiting examples, the constructs shown in FIG. 17).

### 5.4 Methods of Treatment

The present invention provides for methods of treating a vascular disorder comprising altering the activity of Vezf1 in the subject.

In certain embodiments of the invention, the vascular disorder is associated with undesirable angiogenesis, in which case it is desirable to decrease Vezf1 activity in a subject or in a particular tissue or cell population of the subject. Such a decrease may be achieved by either inhibiting the expression of endogenous Vezf1 genes, for example, by the administration of an antisense RNA or ribozyme or an inhibitor of the Vezf1 promoter, or inhibiting the action of Vezf1 protein, for example, by administering an antagonist of Vezf1 (which may be identified using an animal model system described above) or by administering an anti- Vezf1 antibody (or a fragment or derivative thereof). Conditions which may benefit from such treatment include, but are not limited to, neoplasms (where the blood supply to the neoplasm may be compromised), atherosclerosis, post-angioplasty restenosis, diabetic retinopathy, rheumatoid arthritis, hemangiomas, psoriasis, duodenal ulcers, and vascular malformations (Folkman, 1995, Nature Med. 1:27-31).

In other embodiments, the vascular disorder is associated with insufficient angiogenesis, or the primary disorder is one in which increased angiogenesis is desirable. Under such circumstances, the activity of Vezf1 in a subject, or in a particular tissue or cell population of the subject, may be increased. Such an increase may be achieved, for example, by increasing the transcription of Vezf1 by administering an agent which stimulates the Vezf1 promoter, or by increasing the number of Vezf1 genes (e.g. by gene therapy, for example using a retroviral or adenovirus vector comprising a Vezf1 gene or cDNA operably linked to a promoter element). Alternatively, Vezf1 protein may be administered to the subject, or an agonist of Vezf1 activity may be administered. Conditions which may benefit from such treatment include ischemic conditions (associated, for example, with myocardial, brain or peripheral vascular ischemia), wound healing, tissue grafting (including transplant) and conditions involving endothelial cell growth/proliferation, for example after coronary angioplasty, stenting or related procedures, re-endothelialization of arterial grafts, and endothelial regeneration in A-V shunts, e.g. in renal dialysis patients.

In particular embodiments, the present invention provides for a method of increasing angiogenesis in a tissue of a subject in need of such treatment, comprising increasing the amount of Vezf1 activity in the tissue. Nonlimiting examples of such tissues include myocardial tissue, brain tissue, wounded tissue, and grafted tissue.

In alternative embodiments, the present invention provides for a method of decreasing angiogenesis in a tissue of a subject in need of such treatment, comprising decreasing the amount of Vezf1 activity in the tissue. In a specific nonlimiting embodiment, the tissue is a neoplasm, and in particular a malignant neoplasm.

Administration of the foregoing agents may be local or systemic, using a suitable pharmaceutical carrier. Other compounds which aid in the uptake or stability of these agents, or which have beneficial pharmaceutical activity, may also be included in the formulations of the invention.

### 6. EXAMPLE

### Preparation of an Embryonal Stem Cell Clone Having a Retroviral Insertion in Vezf1

### 6.1. Materials and Methods

Retroviral vectors and cells. Retroviral entrapment vectors containing a human alkaline phosphatase (“AP”) reporter gene, namely PT-AP (which requires insertion in the proximity of a cellular promoter for AP expression, and hence is referred to as a “promoter trap vector”), GT-AP (which contains a splice acceptor site 5′ of the AP gene which can combine with a splice donor site from cellular RNA, and hence is referred to as a “gene trap vector”) and PT-IRES-AP (which contains an internal ribosomal entry site upstream of the AP gene, and which can form a bicistronic transcript with a cellular RNA), and Ψ2 packaging lines stably producing the retroviral vectors, were as depicted in FIG. 18. The ES cell line R1 (Nagy et al., 1993, Proc. Natl. Acad. Sci. U.S.A. 90:824-828) was obtained at passage 8 from A. Nagy, Toronto, Canada, and aliquots were prepared and frozen at passage 10. The R1 cells were maintained in ES cell culture medium (high glucose Dulbecco's Modified Eagles Medium (“DMEM”) supplemented with 15 percent heat-inactivated bovine serum (IIyClone); 20 mM HEPES, pH 7.3, 20 mM non-essential amino acids (GIBCO), β-mercaptoethanol (Sigma), penicillin and streptomycin, 500 U/m1 LIF (GIBCO)). The cells were grown in gelatinized dishes on γ-irradiated embryonic fibroblasts as feeder cells that were prepared from day 13 IL-6(−/−) embryos obtained from F. Constantini, Columbia University.

Infection of ES cells with retroviral entrapment vectors.

Infection by co-cultivation with virus producing cells. ψ2 producer clones (5×10⁶ cells per 10 cm dish) stably transfected with the retroviral entrapment construct PT-AP were treated for 3 hours with 10 μg/ml mitomycin C (Boehringer), washed three times with PBS, trypsinized and plated onto 10 cm dishes in ES cell culture medium supplemented with 4 μg/ml polybrene (Sigma). After attachment, about 1×10⁶ R1 cells and embryonic fibroblast feeder cells were plated onto the same dish. After 48 hours co-cultivation, the ES cells were trypsinized, and infected clones were selected for 7 days by plating them in ES cell medium supplemented with 350 μg/ml G418 (GIBCO).

Infection with virus supernatant. R1 cells, plated the preceding day at a density of 1×10⁶ cells per 10 cm dish in ES cell medium supplemented with polybrene (4μg/ml), were infected by adding 2 ml of freshly harvested and filtered (0.4μfilters) supernatant from T2 clones producing GT-AP or PT-IRES-AP virus (multiplicity of infection (“MOI”) between 0.01-0.1). Four hours later, 8 ml of ES cell medium was added. Thirty-six hours after infection, G418 selection medium was applied to the cells. Single g418-resistant colonies were isolated seven days later under a dissection microscope, trypsinized and divided into two parallel 96-well plates. Cells in one 96-well dish (the master plate) were frozen down, whereas cells from its parallel plate were expanded into 24-well dishes for further analysis.

Differentiation of ES cells into embryoid bodies. Individual ES cell clones, grown to 30-50 percent confluency in 24-wekk dishes, were pooled in groups of 6 to 8 and induced to spontaneously differentiate into embryoid bodies essentially as described in Doetschman et al., 1985, J. Embryol. Exp. Morph. 87:27-45 and Lindenbaum and Grosveld, 1990, Genes Dev. 4:2075-2085. Cells were plated in 6 cm gelatin-coated dishes in the absence of feeder cells in ES cell medium without LIF. After two days in culture, small ES cell clumps were lifted off the plates by gentle trypsinization and transferred into suspension culture (DMEM supplemented with 10 percent heat-inactivated fetal bovine serum; 20 mM HEPES, pH 7.3; penicillin and streptomycin) in 6 cm petri dishes (Labtech, Nunc). After four days, “simple embryoid bodies (“EBs”) had formed, consisting of an outer layer of endodermal cells, a basal lamina and an inner layer of columnar, ectodermal cells (Robertson, 1987, in Teratocarcinomnas and embryonic stem cells: a practical approach, Robertson, ed., IRL Press, Oxford, England, pp.71-112). The simple embryoid bodies were kept for another six days in suspension culture, where they further differentiated into complex “cystic embryoid bodies” containing visible cavitation, pockets of primitive blood islands, and rhythmically contracted cardiomyocytes. In parallel, day 4 simple embryoid bodies were re-plated on gelatinized tissue culture plates, where they attached within 24 hours. Outgrowth of differentiated cells with various phenotypes, including fibroblasts, endothelial-like cells, neurons, contracting cardiomyocytes, differentiated cell types with an uncharacterized phenotype, as well as pockets of undifferentiated cells, was observed during the next six days. Aliquots of embryoid bodies at day 4, day 7 and day 10 in suspension culture were harvested, fixed and histochemically stained for human placental alkaline phosphatase activity in the presence of the inhibitor levamisole (0.24 mg/ml) as described in Fields-Berry et al., 1992, Proc. Natl. Acad. Sci. U.S.A. 89:693-697. To inactivate endogenous mouse AP activity, ES cells and embryoid bodies were heat-treated for 30 minutes at 70° C. Day-6 cultures of re-plated embryoid bodies were examined for AP activity as well. Once positive pools were identified, individual clones from these pools were analyzed for AP activity in the form of undifferentiated ES cells, upon embryoid body formation, and in cultures of re-plated embryoid bodies. Only those ES cell clones that displayed reproducible AP expression in at least 50 percent of all embryoid bodies present in each culture sample were scored as positive. Positive ES cell clones were re-tested for their AP expression pattern, expanded, and frozen in aliquots for further analysis.

### 6.2. Results

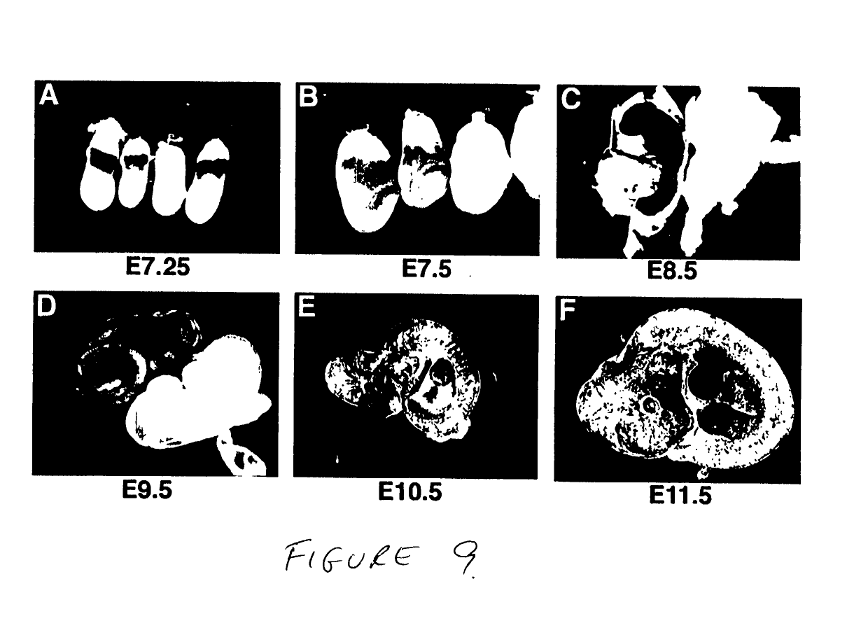

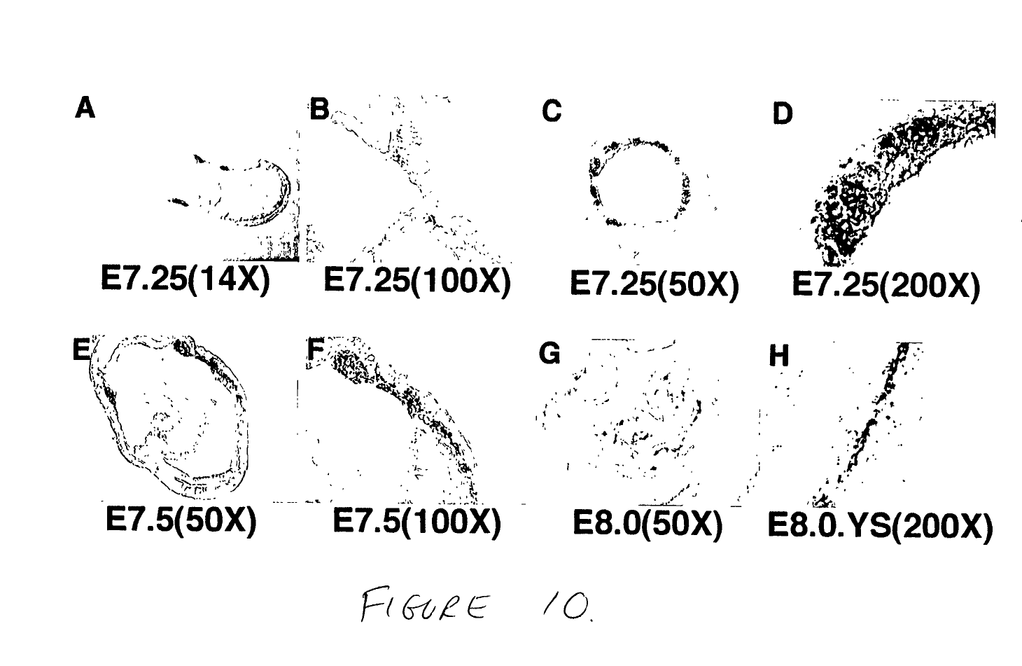

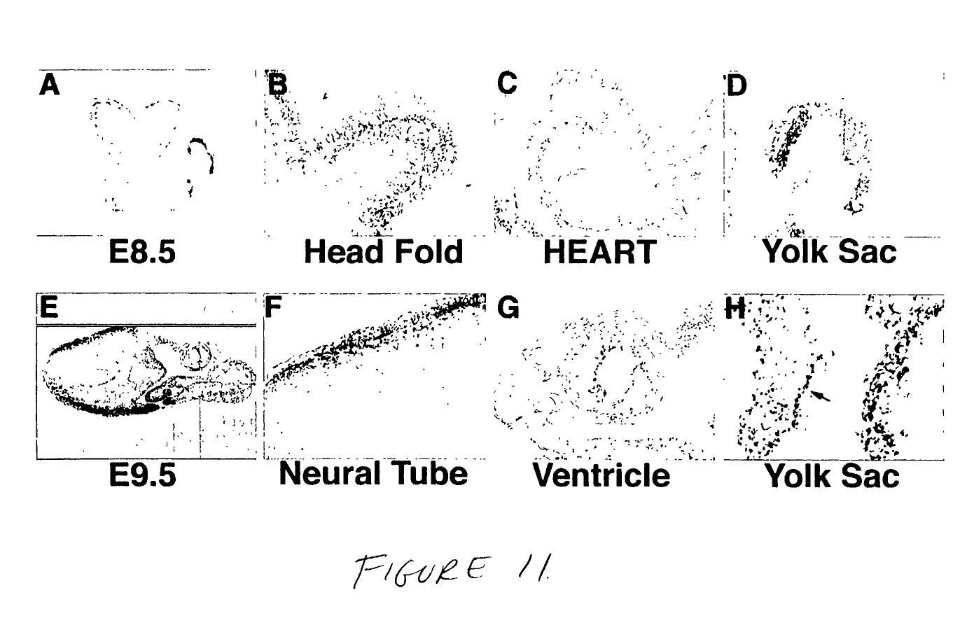

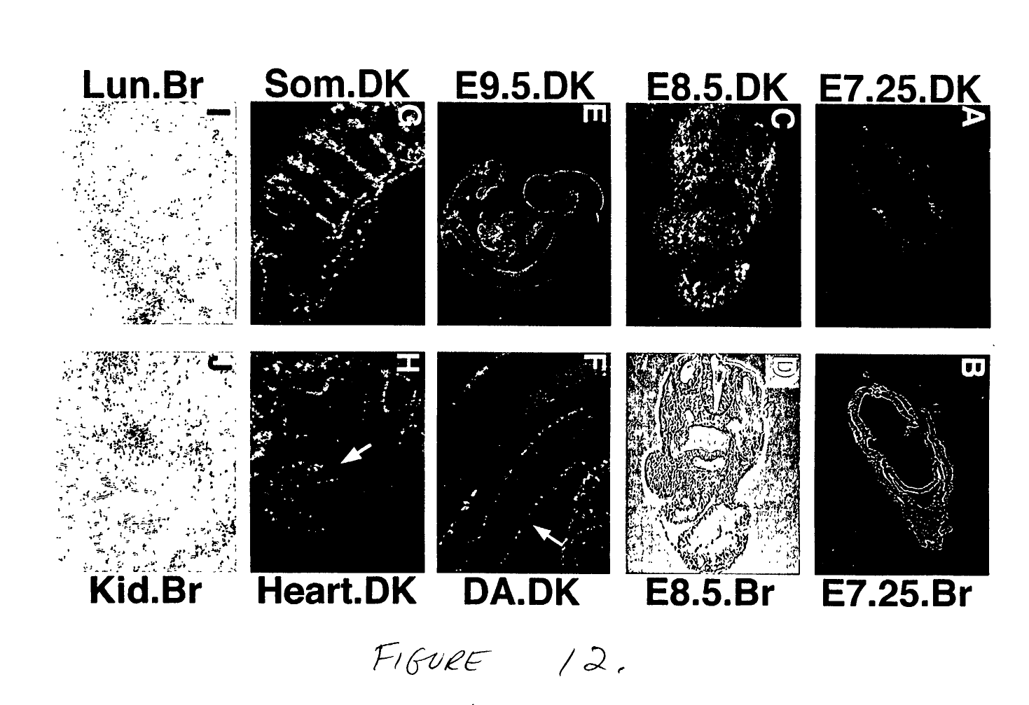

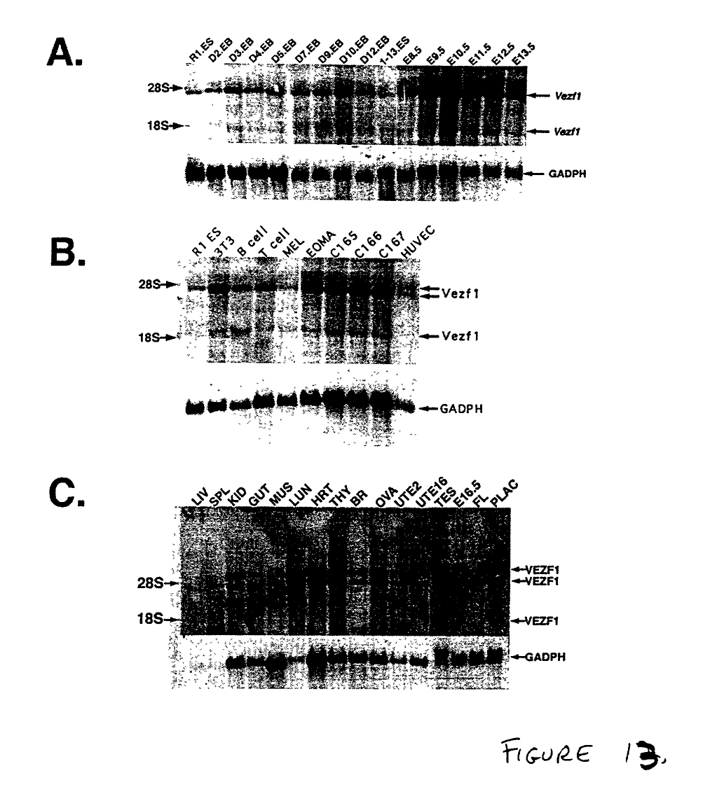

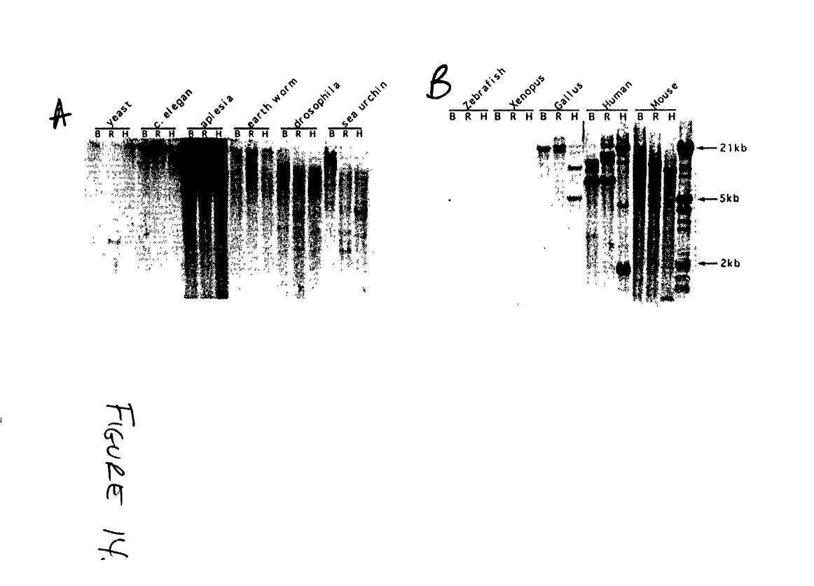

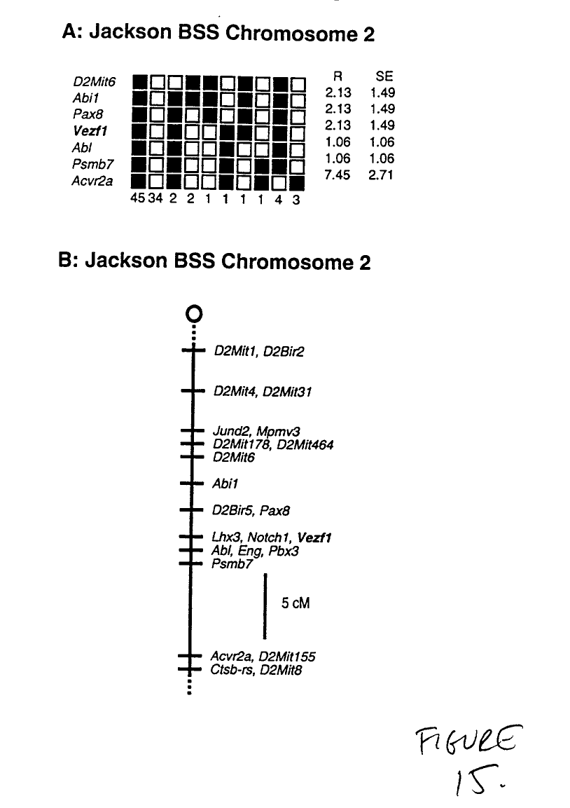

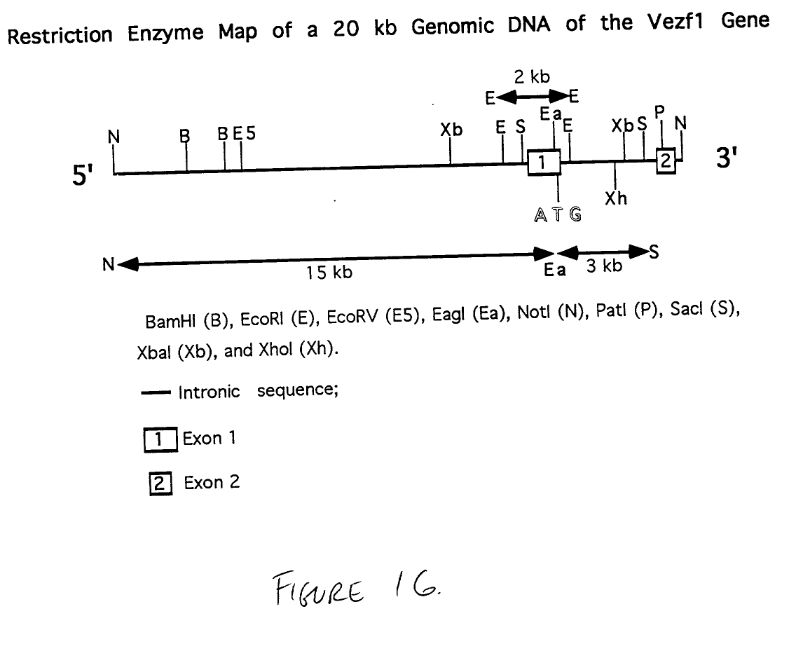

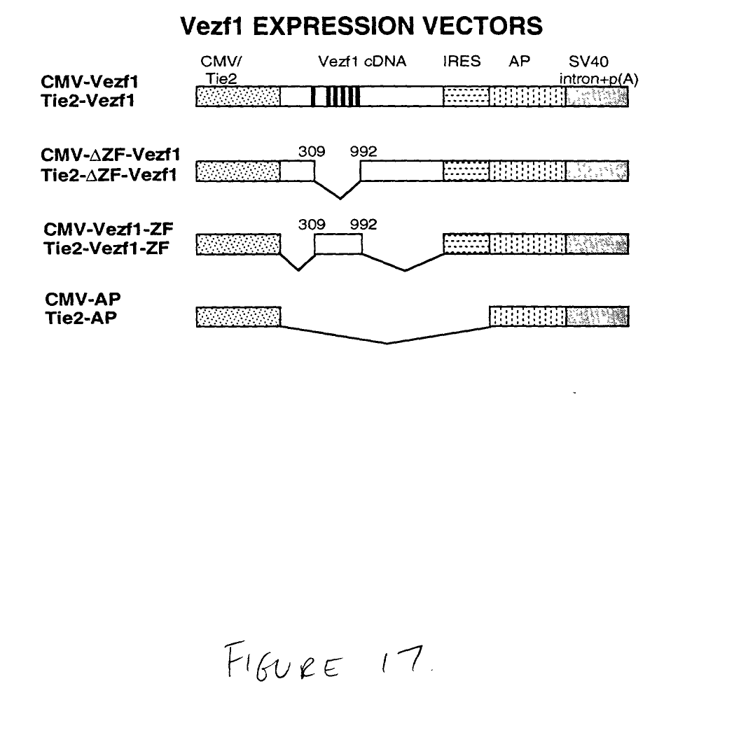

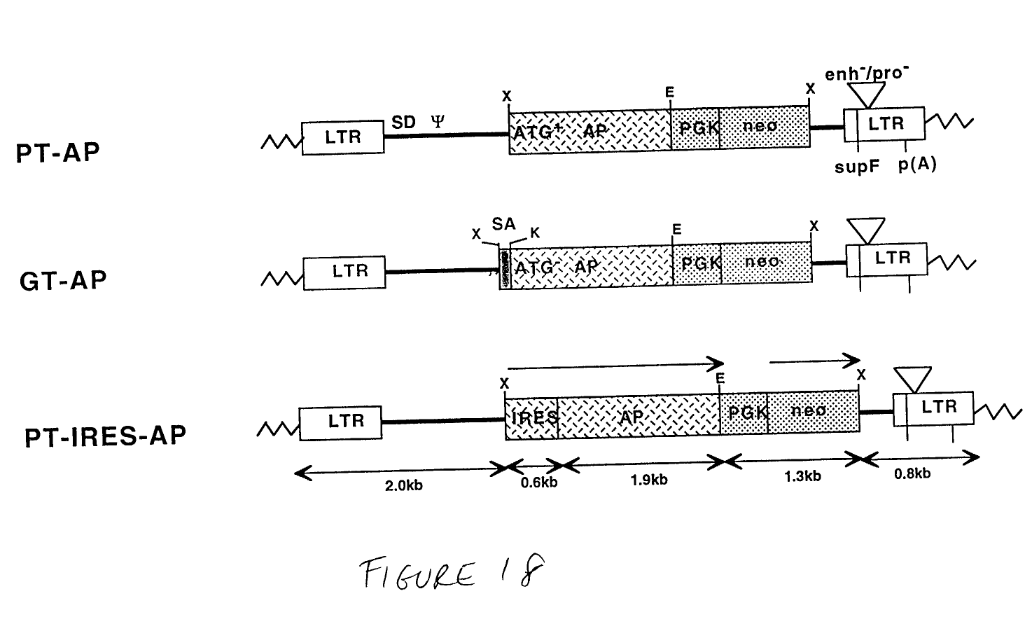

Developmentally regulated genes were sought to be identified using retroviral entrapment vectors, as depicted in FIG. 3. Expression of the AP reporter gene in ES cells with entrapment vector insertions during in vitro differentiation was characteristic and highly reproducible for each individual clone, examples of which are illustrated in FIG. 4A-X. AP staining was visible as a dark purple precipitate that was easily distinguished from the faint pink background in embryoid body cultures derived from uninfected R1 control cells (FIG. 4A,B). Clone PT-AP #IVE3-8 showed weak expression in undifferentiated ES cells, widespread and strong expression in day-4, and weaker, more restricted expression in day-7 and day-10 EBs (FIG. 4C,D). Clone GT-AP #7-65 displayed spotty AP staining exclusively during embryoid body formation, increasing in intensity between day-4 and day-10 cultures (FIG. 4E,F). PT-IRES-AP clones #1-13 and #9-G7 showed spotty expression in embryoid bodies, varying in intensity between day-4 and day-10 cultures (#1-13, FIG. 4G,H; #9-G7, FIG. 4W,X), and restricted expression in re-plated embryoid bodies. In clones PT-IRES-AP #1-58 and #2-86, strong and patchy AP expression was restricted to day-7 and day-10 embryoid body cultures (FIG. 41,J and FIG. 4K,L, respectively). PT-IRES-AP clone #2-51 showed spotty expression in undifferentiated ES cells and day-7 and day-10 embryoid bodies, but not in day-4 embryoid bodies or re-plated embryoid bodies (FIG. 4M,N). Clone PT-IRES-AP #6-33 displayed spotty AP expression restricted to embryoid bodies, increasing in intensity from day-7 to day-10 cultures (FIG. 4Q,R). Finally, PT-IRES-AP clones #4-45, #7-26, and #9E5 stained positive for the AP reporter gene in all stages of in vitro differentiation, including re-plated embryoid bodies, and they displayed either widespread (#7-26, FIG. 4S,T) or patchy AP expression (#4-45, FIG. 4O,P; #9E5, FIG. 4U,V). Clone PT-IRES-AP #1-13 was chosen for further study.

### 7. EXAMPLE:

### Promoter Trap Insertion Into Vezf1

### 7.1. Materials and Methods

Molecular cloning of #1-13 retroviral flanking genomic DNA. Genomic DNA flanking the #1-13 retroviral insertion was cloned by supF complementation (Reik et al., 1985, Proc. Nati. Acad. Sci. U.S.A. 82:1141-1145; Soriano et al., 1987, Genes Dev. 1:366-375). A recombinant DNA library was constructed by ligating EcoRI-cleaved genomic DNA from the #1-13 ES cell clone to the EcoRI site of γZAP vector DNA (Stratagene). The ligation mix was packaged in vitro using Gigapack II Gold packaging extract (Stratagene) and titered on E. coli VCS257 bacteria. About 2×106 plaques were used to infect the supf strain E. coli MCI 060. About two percent (4 out of 200) plaques that grew on MCI 060 bacteria contained the proviral DNA insert, as shown by plaque hybridization with a neo- or AP-specific probe. pBluescript SK phagemids Stratagene containing the cloned insert were rescued from γZAP by in vivo excision, thereby producing a 0.7 kb #1-13 3′ EcoRI flanking genomic DNA region probe.

Characterization of cDNAs. cDNAs corresponding to the #1-13 insertion were isolated using the flanking region probe to screen a day-7.5 mouse embryonic cDNA library (provided by J. Gerhart). Sequencing was performed using a commercial sequencing kit (Sequenase Version 2.0, USB) and synthetic oligonucleotide primers specific for the U3 region (5′ GAG TGA TTG ACT ACC C 3′; SEQ ID NO: 5) or U5 region (5′ GCT AGC TTG CCA AAC C 3′; SEQ ID NO: 6) of the Mo-MLV LTRs and T3 or T7 primers, respectively. Two cDNA clones, termed 10-1 and 10-2, were isolated and sequenced as above. Sequencing homology analysis was performed using the BLAST comparison algorithm (Netscape Navigator, 3.01). Both cDNA clones were found to have significant homology with human DB1 cDNA (Koyano-Nakagawa et al., 1994, Mol. Cell. Biol. 14:5099-5107). Full length cDNA was obtained by RT-PCR of total RNA from day 7 embryoid bodies. The first strand DNA was synthesized in a 25 μl reaction containing 1 μg RNA, 2 pmol primer, 400 μM dNTPs, 10 mM DTT, 1x first strand buffer (Gibcol BRL) and 200 units of Superscript II (Gibcol BRL) at 42° C. for one hour. The primer for the RT reaction was 5′ TCATTGCTATATTGAGA 3′(SEQ ID NO: 7) from Vezf1 cDNA (nucleotides 1487-1503). The cDNA fragment of Vezf1 was amplified in a 100 μl PCR reaction containing 5 μl of RT mixture, 20 pmol of primers, 200 μM dNTPs, 2 units of Vent (NEB), 1x Vent reaction buffer with 2 mM MgSO₄. The PCR primers used were, as 5′ primer, EcoRI.DB1A: 5′ GGAATTCCGGGGGGAGTGGGGAGGA 3′ (SEQ ID NO: 8) from human DB1 cDNA (nucleotides 3-20) and, as 3′ primer, BamHI.12T7.2: 5′ CGGGATCCCGTGATGGTGACAGGGTGTGCTA 3′ (SEQ ID NO: 9) from mouse Vezf1 cDNA (nucleotides 1423-1443). The PCR cycles used were 94° C., 1 minute; 55° C., 1 minute; 72° C., 2 minutes, for 35 cycles. PCR fragments of Vezf1 were subcloned into a pBluescript vector (Stratagene) and subjected to DNA sequencing.

ES cell cultures and embryoid bodies. Clone #1-13 ES cells prepared as described in the preceding section were maintained in an undifferentiated state by growing in gelatinized dishes on γ-irradiated mouse embryonic fibroblast feeder cells. ES cells were induced to differentiate into embryoid bodies in vitro by plating small ES cell clumps in suspension culture for 4-10 days. Day 4 embryoid bodies were re-plated on gelatinized tissue culture plates, and attached embryoid bodies outgrew to differentiate into different cell types, referred to as “replated embryoid bodies”. ES cell culture medium and embryoid body medium were as described in the preceding section.

Generation of ES cell aggregation chimeras and transgenic lines. ES cells were used to generate germ-line chimeras by using the ES cell-diploid embryo aggregation method essentially as described in Nagy and Rossant, 1993, in Gene targeting: a practical approach, Joyner, Ed., Oxford University Press, NY, pp. 147-179.Briefly, ES cells were used to aggregate with 8-cell stage CD-1 mouse embryos, and aggregates were cultured at 37° C. to develop into blastocysts or compacted morula. These were reintroduced into the uterus of day 2.5 pseudo-pregnant Swiss Webster foster mothers and allowed to develop to term. Chimeric males with strong ES cell contribution were identified by their agouti coat color and eye pigmentation. To test germline transmission, chimeric males were bred with CD-1 females, and agouti F1 offspring were examined for the presence of entrapment vector DNA in their tail DNAs. Inbred mice heterozygous for the insertion were generated by crossing with 129/Sv females. Homozygous offspring were obtained by intercross of F1 heterozygous parents. The genotypes of these animals were determined by Southern blot analysis on genomic DNA, using a #1-13 flanking genomic probe.

AP staining in whole-mount embryos and histological sections. Staged embryos were dissected out of the CD-1 females that were mated with heterozygous males carrying the #1-13 retroviral insertion. Mouse embryos and extraembryonic membranes from E7.5 to E13.5 were subjected to AP staining with the BM purple BCIP/NBT substrate (Boehringer Mannheim) in the presence of levamisole (Fields-Berry et al., 1992, Proc. Natl. Acad. Sci. U.S.A. 89:693-697). AP stained whole-mount embryos were embedded in paraffin, and sectioned by conventional methods (Hogan et al., 1994, Manipulating the mouse embryo: a laboratory manual, Cold Spring Harbor Laboratory Press, Cold Spring Harbor, NY). Whole mount and sectioned embryos were photographed under a Nikon SMZ-U stereomicroscope with a digital camera.

RNA in situ hybridization. Clone 10-1 of the Vezf1 cDNA, corresponding to nucleotides 1397 through 2899, was used as a template for both sense and antisense probe synthesis by RNA in vitro transcription. In situ hybridization to sectioned embryos was performed as described in Sassoon and Rosenthal, 1993, Meth. Enzymol. 225:384-404. Birefly, embryoid bodies or staged mouse embryos fixed in 4 percent formaldehyde were embedded in paraffin at 60° C. following a series of dehydrating washed in ethanol and a final 30 min wash in xylene. Material sectioned at 6μm was de-paraffinized and digested with proteinase K followed by a 16 hour hybridization with ³⁵S-radiolabeled antisense RNA probes (final probe concentration 75 dpm/ml in hybridization buffer). Post-hybridization washes of increasing stringency were included to reduce background. Sense control probes were synthesized for Brachyury and Fgf-5. Slides were dipped in Kodak NBT-2 emulsion, dried overnight and exposed for 5 days to 2 weeks at 4° C. Material was counter-stained with toludine blue, unless it was stained before as whole mount for AP, and photographs were taken using a Lica Leitz DMRB Microscope.

RNA isolation and Northern hybridization analysis. Total RNA was prepared from tissues and cell lines by using a modification of the guanidinium-CsC1 method described in Chirgwin et al., 1979, Biochemistry 18:5294-5299. Tissues were homogenized in guanidinium buffer (4M guanidinium thiocyanate, 25 mM Na citrate, pH 7.0, 0.5 percent sarcosyl, 0.1 M β mercaptoethanol). Cultured cells and embryoid bodies were washed in ice cold PBS and lysed in guanidinium buffer, and the viscosity of the lysate was reduced by shearing through a 21-gauge needle. RNA was pelleted through a CsC1 step gradient (6.2 M CsC1, 0.1 M EDTA, pH 8.0) by centrifugation for 18 hours at 35,000 rpm at 15° C. RNA pellets were washed with 80 percent ethanol/TE (where “TE” is 10 mM Tris, pH 7.5, 1 mM EDTA, pH 8.0), dissolved in TE, and precipitated by adding 0.1 volumes of 3M Na acetate and 2.5 volumes of ethanol. 20 μg samples of RNA were electrophoresed on 1 percent agarose-formaldehyde gels using standard procedures. Gels were transferred to nitrocellulose membranes (Optitran, Schleicher & Schuell) in 20X SSC. The filters were hybridized with 1×10⁷ cpm of random hexamer-primed, [α−³² P] dCTP-labeled fragment of the Vezf1 cDNA (nucleotides 3-776) at 42° C. in 50 percent formamide. The filters were washed at 50° C. in 0.2X SSPE (36 mM NaCl, 2 mM NaPO₄, pH 7.7, 0.2 mM EDTA), 0.1 percent SDS and exposed for 1-2 days. To control for RNA loading, filters were stripped and hybridized with a [α−³² P] dCTP-labeled mouse GADPH cDNA plasmid.

Southern analysis of genomic DNA and “zoo blot” analysis. High molecular weight DNA from cultured cells and tail biopsies was prepared as described in Laird et al., 1991, Nucleic Acids Res. 19:4293. Agarose gel electrophoresis and Southern analysis were performed as described in Stuhlmann et al., 1990, J. Virol. 64:5783-5796. For Southern analysis of “zoo blots”, high molecular weight DNA was prepared from various vertebrate and invertebrate species. 20 μg DNA of mouse or human DNA, and equivalent amounts of DNA from other species reflecting their genome complexity, were digested with BamHI, EcoRI or 4HindIII, respectively. Digested DNA was separated on 0.8 percent agarose gels and transferred to nitrocellulose membranes (Optitran, Schleicher & Schuell). The filters were hybridized to 2×10⁷ cpm of a [α−³² P] dCTP-labeled fragment of the Vezf1 cDNA, corresponding to nucleotides 697-1480, including 3½ of the six zinc fingers, under reduced stringency conditions (35° C. in 50 percent formamide, 5X SSC, 5X Denhardt's, 0.5 percent SDS, and 100 μg/ml denatured salmon sperm DNA). Blots were washed at increasing stringency, with the final washes at 65° C. in 0.2X SSC, 0.1 percent SDS for 30 minutes (twice), and exposed for 2-4 days.

Chromosomal mapping. A DNA panel from the Jackson Laboratory community resource (C57B⅙ J×M. spretus) F1×M. spretus interspecific backcross (Rowe et al., 1994, Mamm. Genome 5:253-274) was used to map the Vezf1 locus using PCR techniques. Single strand conformational polymorphism (SSCP) between the two parental strains C57B⅙ J and M. spretus was detected using a pair of primers that span an intron in the 3′ flanking genomic DNA. The PCR primers were 5′ GGAGGTGTTTGTACACATGCTCTG 3′ (Map 3A; SEQ ID NO: 10) and 5′ GCTGTGGATGGAGCAGAATCCAGT 3′ (Map 3B, SEQ ID NO: 11). A 25 μl PCR reaction containing 50 ng DNA template, 1X PCR buffer, 50 μM dNTPs, 0.5 mM Mg²⁺, 10 pM of each primer, 1 unit of Taq polymerase (NEB BioLabs) and 10μCi [α−³² P] dCTP per eight reactions was subjected to 35 cycles of PCR having the conditions 94° C. for 1 minute, 55° C. for 1 minute, and 72° C. for 2 minutes. The PCR products were resolved on a 6 percent non-denatured polyacrylamide gel (Spinardi et al., 1991, ) at 30 W constant power at 4° C. for about 3 hours. Each sample was genotyped for the presence of the M. spretus allele and the results were transmitted back to the database of the Jackson Laboratory for comparison with the existing map.

### 6.2. Results

Identification of the PT1-13 clone during ES cell differentiation. The #1-13 ES cell clone was identified and isolated based on AP reporter gene expression during R1 ES cell differentiation into EBs in vitro (see preceding section and FIG. 5A-D). The #1-13 ES cells tested were either (i) ES cells which had been induced spontaneously to form embryoid bodies in suspension culture, or (ii) re-plated day-4 embryoid bodies in gelatin-coated tissue culture dishes. Aliquots of embryoid bodies in suspension culture at day-4, day-7 and day-10, or re-plated day-4 embryoid bodies were harvested and assayed for AP expression. Spotty purple staining was observed in day-4 simple embryoid bodies (as indicated by the arrow in FIG. 5A) and even more appearent on the surface of transparent cystic bag-like structures in certain advanced day-4 embryoid bodies (as indicated by the arrowhead in FIG. 5A). Stronger purple staining was observed around all day-7 cystic embryoid bodies (FIG. 5B), but less staining was observed in day-10 embryoid bodies (FIG. 5C). Strong purple staining was observed in groups of unidentified cells of the replated day-4 embryoid body monolayers in gelatin-coated dishes (FIG. 5D). Therefore, AP reporter gene expression was detected in the simple embryoid bodies originating from ES clone #1-13, in which several early cell lineage marker genes such as Oct-3, Fgf-5, GATA-4 and Flk-1 are expressed.

Isolating proviral flanking genomic DNA. The retroviral IRES-PT-AP vector used to generate clone #1-13 ES cells contained a unique EcoRI restriction site located between the AP gene and the PGK-neo cassette (see FIG. 3). A 0.7 kb 3′ EcoRI flanking genomic DNA fragment was cloned by supf complementation (see FIG. 6A) and subjected to DNA sequencing. Southern analysis was performed (FIG. 6B) using high molecular weight DNA from R1 ES cells, the #1-13 ES clone, and transgenic lines containing the #1-13 retroviral insertion, using the 0.7 kb fragment of flanking cellular DNA as a probe. In R1 ES cell DNA, only a 8.0 kb endogenous EcoRI fragment of the Vezf1 gene was detected. Clone #1-13 ES cell DNA contained the 8.0 kb endogenous fragment as well as a 2.8 kb fragment which hybridized to the probe. In mice, wild-type (CD-1) DNA contained the 8.0 kb band, DNA from heterozygous mice exhibited bands representing the 8.0 kb endogenous fragment, and the 2.8 kb fragment corresponding to the retroviral insertion. DNA from mice homozygous for the #1-13 insertion lacked the endogenous 8.0 kb fragment. This data indicates that the 0.7 kb genomic fragment isolated by supF complementation is indeed derived from the endogenous gene containing the #1-13 retroviral insertion.

Isolation of Vezf1 cDNA. The 0.7 kb flanking genomic DNA fragment was used as a probe to screen a E7.5 mouse embryo cDNA library. Two classes of overlapping cDNA, as contained in clones 10-1 and 10-2, were isolated. Both share significant homology to DB1. RT-PCR was used to obtain a full-length coding cDNA of Vezf1 by using a 3′ primer derived from clone 10-1 and a 5′ primer from the human DB1 cDNA (see Materials and Methods, supra). A 3,640 bp full-length Vezf1 cDNA was isolated using these two complementary approaches, and was subjected to DNA sequencing to obtain the nucleic acid and deduced amino acid sequences shown in FIG. 1. This gene was named Vezf1, for “vascular endothelial zinc finger”, based on its expression pattern and structural hallmark. An in-frame ATG codon is found in the sequence at a position consistent with its use as an initiation codon, and a typical GC-rich sequence is found in proximity to the 5′ end of the predicted coding sequence. From the deduced amino acid sequence, the predicted Mr of mouse Vezf1 is approximately 56 kd. Vezf1 contians six zinc finger domains of the Cys2/His2 type (FIG. 1, underlined; similar to the zinc finger domains of DB 1, Koyano-Nagakawa et al., 1994, Mol. Cell. Biol. 14:5099-5107), a glutamine stretch (indicated by asterisks in FIG. 1), and periodically appearing proline residues in the carboxy-terminal 100 amino acids. When the deduced amino acid sequence of mouse Vezf1 was aligned with and compared to the human DB1 sequence (Koyano-Nagakawa et al., 1994, Mol. Cell. Biol. 14:5099-5107), it was found that the two proteins share 98 percent sequence identity. Database searches have revealed that mouse Vezf1 has 92 percent amino acid sequence identity over the last five zinc fingers with mouse Pur-1, a homolog of human Maz (ZF87) protein, which specifically binds to the myc promoter. Amino acid sequence alignment of residues 168-360 of mouse Vezf1 (SEQ ID NO: 12) and residues 275-469 of mouse Pur-1 (SEQ ID NO: 13) is shown in FIG. 7.

Retroviral insertion into the 3′UTR of the Vezf1 gene. When the 0.7 kb retroviral-flanking genomic DNA fragment referred to above was sequenced and aligned with Vezf1 cDNA, the retroviral insertion was found to be located in the 3′ untranslated region (UTR) of the Vezf1 gene (FIG. 8).

Transgenic mouse lines carrying the #1-13 retroviral insertion. Clone #1-13 ES cells were used to generate complete mouse embryos by ES cell-morula aggregation techniques, and the embryos were allowed to develop to term to produce chimeric mice. Heterozygous mice carrying the retroviral insertion were obtained by breeding chimeric males with wild type CD-1 or 129/Sv females. Homozygous mice were generated from intercrosses between heterozygous mice carrying the retroviral insertion. Genotypes of offspring were confirmed by Southern analysis of DNA obtained from tail biopsies. No obvious phenotypes were observed in either heterozygous or homozygous mice carrying the insertion, which may be explained by the observation that insertion had occurred in an untranslated region of the gene, resulting in incomplete disruption of its expression.

AP reporter and Vezf1 gene expression.

Expression in hemangioblasts. AP expression was first detected in the extraembryonic mesoderm of the yolk sac as a ring-shaped stripe in whole-mount embryos at about E7.25 (FIG. 9A). From histological sections of E7.25 embryos, restricted AP expression in visceral mesoderm of yolk sac, but not extraembryonic endoderm, was detected (FIG. 10A). No detectable expression was observed in allantois or the embryo proper at this stage. In situ RNA hybridization using a riboprobe corresponding to nucleotides 1397 to 2820 in the cDNA (probe C in FIG. 8) of sectioned E7.25 embryos showed restricted silver grains in the visceral extraembryonic mesoderm (FIG. 11A). It therefore appears that AP expression faithfully represents Vezf1 gene expression in vivo. AP and Vezf1 RNA expression in the yolk sac appeared concomitantly with the beginning of blood island formation.

Expression during early vasculogenesis. As the neural plate formed (at about stage E7.5), strong AP expression expanded in the yolk sac of embryos, and AP expressing cells moved into the embryo proper. No AP staining in endocardial cells was detectable (FIG. 9B), despite the fact that flk-1 RNA expression has been observed at this stage (Yamaguchi et al., 1993, Development 118:489-498). In sectioned E7.5 embryos, strong AP expression was detected in the extraembryonic mesoderm, and no expression was observed in extraembryonic endoderm (FIG. 10 E,F). Some weak AP expression was seen in embryonic mesoderm of the most anterior and posterior regions (FIG. 10E,F). No AP expression was found in the ectoplacental core, allantois, embryonic ectoderm and endoderm (FIGURE 10E,F). At E8.5, very strong AP staining was detected in the whole embryo, strong expression was found in the allantois, and relatively weak expression was detected in yolk sac membranes (FIG. 9C). In sections of E8.5 embryos, more specific AP expression was located in endothelial cells of dorsal aorta, putative angioblast cells in cephalic mesenchyme, endocardial tubes, and vitelline veins (FIG. 12A,D). Careful examination of sections through E8.5 yolk sac membranes revealed AP expressing cells in yolk sac mesoderm lining the perimeter of blood islands. These yolk sac mesoderm cells form the vascular endothelium that surrounds the inner region of hematopoietic stem cells. No detectable AP expression in hematopoietic stem cells was observed (FIG. 12H). In situ hybridization analysis of sections of E8.5 embryos showed intense areas of Vezf1 expression in the cephalic mesenchyme, the labeled inner layer of endothelial cells in the dorsal aorta, and the endocardium (FIG. 11C and 11E). This further confirms that AP expression accurately parallels that of Vezf1.

Expression during angiogenesis. In E9.5 embryos, AP staining was detected in the whole vascular plexus of the embryo proper and in yolk sac membranes from heterozygous embryos (FIG. 9D). No detectable AP staining was observed in wild-type CD-1 embryos. Similar patterns of AP staining of vascular plexus were observed from E10.5 to E13.5, in which the expression of AP was observed to decrease over time (FIG. 9E and 9F). In sections of E9.5 embryos, AP expression in head mesenchyme showed small clumps of stained cells which may be putative angioblasts. Endothelial cells of the dorsal aorta and its derivatives, such as vessels in the branchial arches and intersomites, endothelial cells forming capillaries, and the endocardium showed strong AP staining (FIG. 12E-H). The endothelial cell-specific AP expression was consistent with Vezf1 gene expression during mouse embryonic angiogenesis (FIG. 11E-J). In sections of E9.5 embryos, high levels of Vezf1 expression were detected in the putative angioblasts in head mesenchyme, the meningeal plexus surround the brain and spinal cord (FIG. 11E,F), the endocardium (FIG. 11H), endothelial cells of the dorsal aorta (FIG. 11F), intersomites (FIG. 11G), the branchial arches, vitelline veins, and blood vessels in mesonephrons and lung and liver rudiments (FIG. 11I,J). In sections of E13.5 embryos, Vezf1 expression was observed in the endothelial cells of vascular plexi.

No detectable AP expression was observed in either erthroid precursors in the yolk sac, in nucleated blood cells from all stages of embryos tested, or in E13.5 fetal liver other than what appeared to be the endothelial lining of capillaries. The data therefore indicates that Vezf1 expression is mainly confined to vascular endothelial cells and their precursors rather than hematopoietic cells.

Vezf1 transcripts. To determine the size, temporal and cell-type specific expression of Vezf1 transcripts, Northern analysis was performed using RNA harvested from staged embryos, embryoid bodies, cell lines, and tissues from adult mice using a mouse Vezf1 cDNA fragment extending from nucleotide 1 to 750 as a probe (probe A in FIG. 8). Hybridization to a GADPH probe was used to standardize the RNA amounts and to establish the integrity of the RNA used (FIG. 13A, 13B and 13C). A major 4.0 kb transcript and a low abundance 1.0 kb transcript were detected in embryonic stages E8.5- E13.5, R1 ES cells and embryoid bodies, and all mouse hematopoietic cell lines and endothelial cell lines tested (FIG. 13A and 13B). ES cells had less Vezf1 expression than embryoid bodies, and no significant changes in Vezf1 gene expression were observed in different stages of embryoid bodies or in different mouse embryo stages. All mouse endothelial cell lines tested had the same transcripts as mouse embryos (FIG. 13B). Among hematopoietic cell lines, T cell and B cells lines had more abundant Vezf1 transcripts than a mouse erythroleukemia (“MEL”) cell line or macrophages (FIG. 13B), which is consistent with observed human DB1 expression in Jurkat Tcells (Koyanao-Nakagawa et al., 1994, Mol. Cell. Biol. 14:5099-5107). Human umbilical vein endothelial cells (“HUVEC”) were found to have a major 4.0 kb transcript as well as a low abundance 4.5 kb transcript (FIG. 13B), consistent with previously observed DB1 expression in HUVEC cells (Koyanao-Nakagawa et al., 1994, Mol. Cell. Biol. 14:5099-5107).

Three Vezf1 transcripts, two abundant transcripts of 4.0 kb and 4.5 kb, and a low abundance transcript of 1.0 kb, among adult tissues of CD-1 mice (FIG. 13C). The 4.0 kb and 4.5 kb transcripts exhibited different distribution among adult tissues. In the heart, thymus, brain and ovary, these two transcripts exhibited approximately equal distribution. In other tissues, such as the kidney, gut, lung, uterus, and testis, the 4.0 kb transcript was found to be more abundant than the 4.5 kb transcript. Furthermore, Vezf1 expression in the uterus showed no changes during pregnancy, as shown by the Northern analysis of uteri at day 2.5 or day 16.5 of pregnancy (lanes labeled UTE2 and UTE16 in FIG. 13C). In E16.5 embryos, no increased Vezf1 expression was observed in the fetal liver (lane labeled FL in FIG. 13C), and there was increased expression in the placenta (lane labeled PLAC in FIG. 13C) which may be attributed to the vascularity of placental tissue. The fact that the Vezf1 cDNA characterized above has 3645 nucleotides, and is therefore smaller than the 4.0 kb transcript detected by Northern analysis, may be due to additional 5′ UTR sequences in the endogenous transcript.

Conservation of Vez1 among species. “Zoo blot” analysis was used to determine whether Vezf1-related genes were present in various vertebrate and invertebrate species. Genomic DNA from yeast, C. elegans, aplysia, earthworm, Drosophila, sea urchin, zebrafish, Xenopus laevis, chicken (Gallus), mouse and human was digested with BamHI, EcoRI, or HindIII, respectively, and was resolved by agarose gel electrophoresis and transferred to nitrocellulose membrane. The resulting blots were hybridized under low stringency conditions to a Vezf1 cDNA probe corresponding to nucleotides 697-1444 (probe B in FIG. 8). Strongly hybridizing fragments were found in chicken, mouse, and human genomic DNA (FIG. 14B). No significant signals were detected in other DNAs (FIG. 14A and 14B). Two highly related genes appeared to be present in chicken, mouse and human genomes, and, in human DNA, several additional sequences with reduced homology were present. These results suggest that Vezf1 is a member of a small family of genes which are conserved in higher vertebrates. The Vezf1-related sequences were only detected in those species with yolk sac membrane, which is consistent with the observed first appearance of Vezf1 gene expression during embryogenesis. Thus, Vezf1 may serve an important function during yolk sac formation.

Chromosomal mapping of the Vezf1 locus. A DNA panel obtained from the Jackson Laboratory community resource (C57BL/6J×M. spretus)F1×M. spretus) interspecific backcross (Rowe et al., 1994, Mamm. Genome 5:253-274) was used to map the Vezf1 locus using PCR techniques. A haplotype figure from the Jackson BSS backcross ((C57BL/6JEi×SPRET/Ei) ×SPRET/Ei) indicated that the proximal region of chromosome 2 contains loci linked to Vezf1 (FIG. 15A and 15B). Vezf1 was found to cosegregate with Lhx3, a limcontaining homeoprotein, and genes encoding mouse Notch 1, retinoid receptor alpha subunit (Rxrα), and brain alpha spectrin (Spnα2). Vezf1 maps adjacent to Abelson proto-oncogene (Ab1), adenylate kinase-1 (Ak1), endoglin (Eng), erythrocyte band 7.2 (Epb7.2), 78 kd glucose regulated protein (grp78), gelsolin (Gsn), complement component C5 (Hc), and a proto-oncogene homeobox gene (Pbx3). This region of the mouse genome has extensive homology to human chromosome 9q. Indeed, most of the above-mentioned genes were mapped to human chromosome 9q, and some of them have been associated with inherited diseases and disorders. For example, the endoglin gene is associated with hereditary haemorrhagic telangiectasia type 1, an autosomal dominant vascular dysplasia (McAllister et al., 1994, Nature Genet. 8:345-351). However, a human EST clone corresponding to the human DB1 gene has recently been mapped to chromosome 22g. This region is known to map several human vascular and hematopoietic disorders.

Various publication are cited herein, the contents of which are hereby incorporated by reference in their entireties.

What is claimed is:

1. A purified and isolated Vezf1 gene having the nucleic acid sequence set forth in FIG. 1 (SEQ ID NO: 1).

2. The Vezf1 gene of claim 1, as contained in a vector molecule.

3. A purified and isolated nucleic acid encoding a Vezf1 protein, wherein the Vezf1 protein has an amino acid sequence as set forth in FIG. 1 (SEQ ID NO: 2).

4. The nucleic acid of claim 3, contained in an expression vector and operably linked to a promoter element.

5. The nucleic acid of claim 4, wherein the promoter element is not the Vezf1 promoter.

6. An expression vector containing the DB1 gene operably linked to a heterologous promoter element, wherein the promoter element is selectively active in vascular endothelial cells.

7. A non-human animal in which one or both endogenous Vezf1 alleles has been altered by homologous recombination with an exogenously introduced nucleic acid.

8. A non-human transgenic animal carrying a transgene which encodes a Vezf1 protein.

9. A non-human transgenic animal carrying a transgene which encodes a mutated Vezf1 protein.

10. A non-human transgenic animal carrying a transgene which is the nucleic acid of claim 1.

11. A non-human transgenic animal carrying a transgene which is the nucleic acid of claim 3.

12. A non-human transgenic animal carrying a transgene which is the nucleic acid of claim 4.

13. A non-human transgenic animal carrying a transgene which is the nucleic acid of claim 5.

14. A non-human transgenic animal carrying a transgene which is the nucleic acid of claim 6.

15. A method of diagnosing a vascular disorder in a subject, comprising measuring the amount of a Vezf1 gene product, where the gene product is selected from the group consisting of RNA and DNA, in a test sample taken from the subject and comparing that amount to the amount of Vezf1 gene product in a matched control sample, wherein a difference in the amount of Vezf1 gene product in the test sample and the control sample correlates with the presence of a vascular disorder in the subject.

16. A method of diagnosing a heritable vascular disorder in a subject, comprising characterizing a Vezf1 gene in the subject and comparing the characteristics of the gene to the normal Vezf1 gene, where a difference in characteristics of the Vezf1 gene in the subject and the normal Vezf1 gene correlates with the presence of a heritable vascular disorder in the subject.

17. The method of claim 15, wherein the vascular disorder is a vascularized neoplasm.

18. A method of increasing angiogenesis in a tissue of a subject in need of such treatment, comprising increasing the amount of Vezf1 activity in the tissue.

19. The method of claim 18, wherein the tissue is myocardial tissue.

20. The method of claim 18, wherein the tissue is brain tissue.

21. The method of claim 18, wherein the tissue contains a wound.

22. The method of claim 18, wherein the tissue is a graft.

23. A method of decreasing angiogenesis in a tissue of a subject in need of such treatment, comprising decreasing the amount of Vezf1 activity in the tissue.

24. The method of claim 23, wherein the tissue is a neoplasm.

25. A method of identifying an endothelial cell, comprising identifying the expression of a molecule selected from the group consisting of a Vezf1-encoding RNA or a Vezf1 protein in the cell.
